(19) United States
(12) Patent Application Publication
(10) Pub. No.: US 2014/0275684 A1
(43) Pub. Date: Sep. 18, 2014

(54) HYDROCARBON TRANSFORMATIONS USING CARBOCATALYSTS

(71) Applicants: Christopher W. Bielawski, Austin, TX (US); Daniel R. Dreyer, Austin, TX (US); Richard Miller, Portola Valley, CA (US)

(72) Inventors: Christopher W. Bielawski, Austin, TX (US); Daniel R. Dreyer, Austin, TX (US); Richard Miller, Portola Valley, CA (US)

(73) Assignee: Graphea, Inc., Austin, TX (US)

(21) Appl. No.: 14/119,808

(22) PCT Filed: May 23, 2012
(86) PCT No.: PCT/US2012/039143

§ 371 Date: Apr. 9, 2014

Related U.S. Application Data
(63) Continuation-in-part of application No. PCT/US2011/038334, filed on May 27, 2011.
Application No. PCT/US2011/038334 is a continuation-in-part of application No. PCT/US2011/038327, filed on May 27, 2011.
(60) Provisional application No. 61/564,126, filed on Nov. 28, 2011.
(60) Provisional application No. 61/564,135, filed on Nov. 28, 2011.
(60) Provisional application No. 61/523,059, filed on Aug. 12, 2011.
(60) Provisional application No. 61/502,390, filed on Jun. 29, 2011.
(60) Provisional application No. 61/496,326, filed on Jun. 13, 2011.

(51) Int. Cl. C07C 6/10; C07C 2/76; C07C 4/06; C07C 5/333

(52) U.S. Cl. CPC C07C 6/10; C07C 5/333; C07C 2/76; C07C 4/06
USPC 585/653; 585/654; 585/700; 585/708; 585/752

(57) ABSTRACT

The disclosure relates to catalytically active carbocatalysts, e.g., a graphene oxide or graphite oxide catalyst suitable for use in a variety of high value chemical transformations.

### CROSS-REFERENCE

This application claims the benefit of PCT International Application No. PCT/US2011/038334, filed May 27, 2011, PCT International Application No. PCT/US2011/038327, filed May 27, 2011, U.S. Provisional Application No. 61/496,326, filed Jun. 13, 2011, U.S. Provisional Application No. 61/502,390, filed Jun. 29, 2011, U.S. Provisional Application No. 61/523,059, filed Aug. 12, 2011, U.S. Provisional Application No. 61/564,135, filed Nov. 28, 2011, and U.S. Provisional Application No. 61/564,126, filed Nov. 28, 2011, which are incorporated herein by reference in their entireties.

### STATEMENT AS TO FEDERALLY SPONSORED RESEARCH AND FUNDING

At least a portion of this invention was made using funding from the Robert A. Welch Foundation under Contract number F-1621.

### BACKGROUND OF THE INVENTION

Cost of goods and services required for manufacture of certain high value organic chemicals is prohibitively high and/or is dependent on starting materials that are by-products of the petroleum industry.

### SUMMARY OF THE INVENTION

Described herein are methods and processes having broad synthetic utility that are applicable for synthesis of certain high value and high volume chemicals that are utilized for a wide range of manufacturing processes. The chemical hydrocarbon transformations provided herein provide cheap and efficient syntheses of certain hydrocarbon raw materials that are used for production of a range of chemical products. Provided herein are methods for synthesis of low molecular weight saturated or partially saturated organic compounds. In some cases, the compounds are gases and/or are volatile compounds.

Provided herein are processes for dehydrogenation of hydrocarbons, cracking of hydrocarbons, coupling of hydrocarbons (e.g., C—C coupling of alkanes), and/or metathesis of hydrocarbons, to provide hydrocarbon products. The processes for dehydrogenation of hydrocarbons, cracking of hydrocarbons, coupling of hydrocarbons (e.g., C—C coupling of alkanes), and/or metathesis of hydrocarbons described herein comprise contacting hydrocarbons with a carbon-based material which is catalytically active. In other words, the synthetic methods described herein comprise the use of catalytically active carbo catalysts. In certain embodiments, the catalytically active carbocatalysts are graphene oxide-derived or graphite oxide-derived catalysts.

In aspect, provided herein is a process for converting
(a) an alkane starting material to an alkene product (dehydrogenation);
(b) an alkane starting material to one or more higher molecular weight alkane products (coupling);
(c) an alkane starting material to one or more metathesis products (metathesis);
(d) a higher alkane starting material to one or more lower alkane products (cracking);
(e) a cycloalkane starting material to an alkane product or an alkene product or a combination thereof (cracking);
or any combination thereof;
   comprising contacting any starting material in (a)-(e) with a catalytically active surface-modified graphene oxide or graphite oxide to provide the corresponding product in (a)-(e).

In one embodiment, provided herein is a process for converting an alkane starting material to an alkene product (dehydrogenation) comprising contacting an alkane starting material with a catalytically active surface-modified graphene oxide or graphite oxide to provide the corresponding alkene product. In one embodiment, provided herein is a process for converting an alkane starting material to one or more higher molecular weight alkane products (coupling) comprising contacting an alkane starting material with a catalytically active surface-modified graphene oxide or graphite oxide to provide the corresponding higher molecular weight product. In one embodiment, provided herein is a process for converting an alkane starting material to one or more metathesis products comprising contacting an alkane starting material with a catalytically active surface-modified graphene oxide or graphite oxide to provide the corresponding one or more metathesis products. In one embodiment, provided herein is a process for converting a higher alkane starting material to one or more lower alkane products (cracking) comprising contacting a higher alkane starting material with a catalytically active surface-modified graphene oxide or graphite oxide to provide the corresponding lower alkane product. In one embodiment, provided herein is a process for converting a cycloalkane starting material an alkane or alkene product or a combination thereof (cracking) comprising contacting a cycloalkane starting material with a catalytically active surface-modified graphene oxide or graphite oxide to provide the corresponding alkane or alkene product or a combination thereof. In one embodiment is a process comprising cracking and coupling. In embodiment is a process comprising cracking and dehydrogenation. In one embodiment is a process comprising metathesis and dehydrogenation.

In one embodiment of the processes above, the catalytically active surface-modified graphene oxide or graphite oxide is catalytically active surface-modified graphene oxide.

In one embodiment of the processes above, the catalytically active surface-modified graphene oxide or graphite oxide is catalytically active surface-modified graphite oxide.

In one embodiment of the processes above, the catalytically active surface-modified graphene oxide or graphite oxide is characterized by one or more FT-IR features at about 3150 cm⁻¹, 1685 cm⁻¹, 1280 cm⁻¹, or 1140 cm⁻¹.

In some embodiments, the catalytically active surface-modified graphene oxide or graphite oxide has a surface modification comprising one or more of a hydrogen peroxide-terminated surface, an epoxide-terminated surface, a ketone-terminated surface, a diketone terminated surface, an aldehyde terminated surface, a carboxyl terminated surface, a hydroxyl terminated surface, an alcohol terminated surface, an ether terminated surface, a dioxirane terminated surface, a quinone terminated surface, a peroxy acid terminated surface, an ester terminated surface, an anhydride terminated surface or a perester terminated surface.

In some embodiments, the catalytically active surface-modified graphene oxide or graphite oxide has a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, or an ether terminated surface.

In some embodiments, the catalytically active surface-modified graphene oxide or graphite oxide has a surface modification comprising one or more of an epoxide-terminated surface, a ketone-terminated surface, an aldehyde terminated surface, a carboxyl terminated surface, a hydroxyl terminated surface, or an alcohol terminated surface.

In a further embodiment of the process, the catalytically active surface-modified graphene oxide or graphite oxide has at least about 25% carbon and at least about 0.01% oxygen as measured by x-ray photoelectron spectroscopy (XPS).

In some embodiments, the carbon-to-oxygen ratio for the catalytically active surface-modified graphene oxide or graphite oxide is between about 1.5:1 and about 1:1.5 as measured by x-ray photoelectron spectroscopy (XPS).

In some embodiments, the carbon-to-oxygen ratio for the catalytically active surface-modified graphene oxide or graphite oxide is between about 1:1 and about 5:1 as measured by x-ray photoelectron spectroscopy (XPS).

In some embodiments, the carbon-to-oxygen ratio for the catalytically active surface-modified graphene oxide or graphite oxide is between about 1:1 and about 4:1 as measured by x-ray photoelectron spectroscopy (XPS).

In some embodiments, the carbon-to-oxygen ratio for the catalytically active carbocatalyst is between about 1:1 and about 3:1 as measured by x-ray photoelectron spectroscopy (XPS).

In some embodiments, the carbon-to-oxygen ratio for the catalytically active surface-modified graphene oxide or graphite oxide is between about 2:1 and about 3:1 as measured by x-ray photoelectron spectroscopy (XPS).

Also provided herein is a process for converting an alkane to an alkene under kinetic control comprising contacting an alkane with a catalytically active surface-modified graphene oxide or graphite oxide described above and herein at a reduced temperature compared to a temperature which is used for the conversion of an alkane to an alkene in the presence of a catalyst which is not a carbocatalyst (e.g., transition metal based catalyst).

Also provided herein is a process for converting an alkane to an alkene under kinetic control comprising contacting an alkane with a catalytically active surface-modified graphene oxide or graphite oxide described above and herein at a reduced temperature compared to a temperature which is used for the conversion of an alkane to an alkene in the presence of a catalyst which is not a catalytically active surface-modified graphene oxide or graphite oxide. In one embodiment of such a kinetically controlled reaction, the reaction provides a higher yield of the alkene and reduces the reverse reaction of hydrogenation of the alkene.

In one embodiment of the kinetic process, the catalytically active surface-modified graphene oxide or graphite oxide is catalytically active surface-modified graphene oxide.

In one embodiment of the kinetic process, the catalytically active surface-modified graphene oxide or graphite oxide is catalytically active surface-modified graphite oxide.

Provided herein is a process for converting ethane to ethene (an exemplary dehydrogenation) comprising contacting ethane with a catalytically active surface-modified graphene oxide or graphite oxide described above and herein.

Also provided herein is a process for converting methane to ethane (an exemplary coupling) comprising contacting methane with a catalytically active surface-modified graphene oxide or graphite oxide described above and herein. Also provided herein is a process for converting methane to propane comprising contacting methane with a catalytically active surface-modified graphene oxide or graphite oxide described above and herein.

Further provided herein is a process for converting ethane to propane (an exemplary metathesis) comprising contacting ethane with a catalytically active surface-modified graphene oxide or graphite oxide described above and herein. Also provided herein is a process for converting methane to butane comprising contacting methane with a catalytically active surface-modified graphene oxide or graphite oxide described above and herein. Further provided herein is a process for converting ethane to butane comprising contacting ethane with a catalytically active surface-modified graphene oxide or graphite oxide described above and herein.

Also provided herein are processes for alkane and/or cycloalkane cracking. Provided herein is a process for converting hexane to methane comprising contacting hexane with a catalytically active surface-modified graphene oxide or graphite oxide described above and herein. Further provided herein is a process for converting hexane to ethane comprising contacting hexane with a catalytically active surface-modified graphene oxide or graphite oxide described above and herein. Also provided herein is a process for converting hexane to propane comprising contacting hexane with a catalytically active surface-modified graphene oxide or graphite oxide described above and herein. Further provided herein is a process for converting hexane to propene (an exemplary cracking and dehydrogenation) comprising contacting hexane with a catalytically active surface-modified graphene oxide or graphite oxide described above and herein. Also provided herein is a process for converting hexane to butane comprising contacting hexane with a catalytically active surface-modified graphene oxide or graphite oxide described above and herein. Further provided herein is a process for converting hexane to butene or butadiene comprising contacting hexane with a catalytically active surface-modified graphene oxide or graphite oxide described above and herein. Also provided herein is a process for converting butane to 1-butene comprising contacting butane with a catalytically active surface-modified graphene oxide or graphite oxide described above and herein. Further provided herein is a process for converting butane to 2-butene comprising contacting butane with a catalytically active surface-modified graphene oxide or graphite oxide described above and herein. Also provided herein is a process for converting butane to butadiene comprising contacting butane with a catalytically active surface-modified graphene oxide or graphite oxide described above and herein.

Further provided herein is a method for alkane metathesis comprising contacting the alkane with a catalytically active surface-modified graphene oxide or graphite oxide described above and herein. In some embodiments, the alkane is a C₁-C₆ alkane and the product obtained from contacting the C₁-C₆ alkane with a catalytically active surface-modified graphene oxide or graphite oxide is a mixture of higher and/or lower C₁-C₁₂ alkanes.

Also provided herein is a method for dehydrogenation of an alkane comprising contacting the alkane with a catalytically active surface-modified graphene oxide or graphite oxide described above and herein. In certain embodiments, the alkane is a C₂-C₆ alkane and the product obtained from contacting the C₂-C₆ alkane with a catalytically active surface-modified graphene oxide or graphite oxide is a mixture of one or more C₂-C₆ alkenes. In some embodiments, the alkane is a C₄-C₆ alkane and the product obtained from contacting the C₄-C₆ alkane with a catalytically active surface-modified graphene oxide or graphite oxide is a mixture of one or more C₄-C₆ dienes.

Provided herein is a reaction vessel comprising the starting materials, the product and the catalytically active surface-modified graphene oxide or graphite oxide described above and herein. In some of such embodiments, the reaction vessel further comprises a source of heat to heat the reaction vessel to a desired temperature, a device for controlling the temperature of the reaction vessel and a device for determining the temperature within the reaction vessel.

In some embodiments, the reaction vessel is in the form of a fluidized bed reactor. In some embodiments, the reaction vessel is in the form of a fixed bed reactor.

In some of the preceding embodiments, the reaction vessel further comprises a solid acid catalyst.

Further provided herein is a catalytically active surface-modified graphene oxide or graphite oxide in contact with ethane and ethene. Further provided herein is a catalytically active surface-modified graphene oxide or graphite oxide in contact with butane and butane (including one or more of 1-butene, 2-butene, butadiene).

Also provided herein is a reaction vessel comprising ethane, ethene and a catalytically active surface-modified graphene oxide or graphite oxide.

In a different aspect, provided herein is a process for converting ethane to ethene comprising contacting ethane with a catalytically active surface-modified graphene oxide or graphite oxide to provide ethene.

In one embodiment of the process, the catalytically active surface-modified graphene oxide or graphite oxide is catalytically active surface-modified graphene oxide.

In one embodiment of the process, the catalytically active surface-modified graphene oxide or graphite oxide is catalytically active surface-modified graphite oxide.

In one embodiment the catalytically active surface-modified graphene oxide or graphite oxide is characterized by one or more FT-IR features at about 3150 cm⁻¹, 1685 cm⁻¹, 1280 cm⁻¹, or 1140 cm⁻¹.

In one embodiment, the catalytically active surface-modified graphene oxide or graphite oxide has a surface modification comprising one or more of a hydrogen peroxide-terminated surface, an epoxide-terminated surface, a ketone-terminated surface, a diketone terminated surface, an aldehyde terminated surface, a carboxyl terminated surface, a hydroxyl terminated surface, an alcohol terminated surface, an ether terminated surface, a dioxirane terminated surface, a quinone terminated surface, a peroxy acid terminated surface, an ester terminated surface, an anhydride terminated surface or a perester terminated surface.

In one embodiment, the catalytically active surface-modified graphene oxide or graphite oxide has a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, or an ether terminated surface.

In one embodiment, the catalytically active surface-modified graphene oxide or graphite oxide has a surface modification comprising one or more of an epoxide-terminated surface, a ketone-terminated surface, an aldehyde terminated surface, a carboxyl terminated surface, a hydroxyl terminated surface, or an alcohol terminated surface.

In one embodiment, the catalytically active surface-modified graphene oxide or graphite oxide has at least about 25% carbon and at least about 0.01% oxygen as measured by x-ray photoelectron spectroscopy (XPS).

In one embodiment, the carbon-to-oxygen ratio for the catalytically active surface-modified graphene oxide or graphite oxide is between about 1.5:1 and about 1:1.5 as measured by x-ray photoelectron spectroscopy (XPS).

In one embodiment, the carbon-to-oxygen ratio for the catalytically active surface-modified graphene oxide or graphite oxide is between about 1:1 and about 5:1 as measured by x-ray photoelectron spectroscopy (XPS).

In one embodiment, the carbon-to-oxygen ratio for the catalytically active surface-modified graphene oxide or graphite oxide is between about 2:1 and about 3:1 as measured by x-ray photoelectron spectroscopy (XPS).

Also provided herein is a process for converting ethane to ethene under kinetic control comprising contacting ethane with a catalytically active surface-modified graphene oxide or graphite oxide described above at a reduced temperature compared to a temperature which is used for the conversion of ethane to ethene in the presence of a catalyst which is not a catalytically active surface-modified graphene oxide or graphite oxide.

Provided herein is ethene prepared according to the process described above.

In another aspect, provided herein is process for converting
(a) a saturated hydrocarbon moiety on a hydrocarbon starting material to an unsaturated hydrocarbon moiety on a hydrocarbon product (dehydrogenation);
(b) a hydrocarbon starting material to one or more higher molecular weight hydrocarbon products (coupling);
(c) a hydrocarbon starting material to one or more metathesis products (metathesis);
(d) a higher hydrocarbon starting material to one or more lower hydrocarbon products (cracking);
or any combination thereof;
   comprising contacting any starting material in (a)-(d) with a catalytically active carbocatalyst to provide the corresponding product in (a)-(d).

In one embodiment, provided herein is a process for converting a saturated hydrocarbon moiety on a hydrocarbon starting material to an unsaturated hydrocarbon moiety on a hydrocarbon product (dehydrogenation) comprising contacting a saturated hydrocarbon moiety on a hydrocarbon starting material starting material with a catalytically active carbocatalyst to provide the corresponding product having an unsaturated hydrocarbon moiety on the hydrocarbon product.

In one embodiment, provided herein is a process for converting a hydrocarbon starting material to one or more higher molecular weight hydrocarbon products (coupling) comprising contacting a hydrocarbon starting material with a catalytically active carbocatalyst to provide the corresponding one or more higher molecular weight products.

In one embodiment, provided herein is a process for converting a hydrocarbon starting material to one or more metathesis products (metathesis) comprising contacting a hydrocarbon starting material with a catalytically active carbocatalyst to provide the corresponding one or more metathesis products.

In one embodiment, provided herein is a process for converting a higher hydrocarbon starting material to one or more lower hydrocarbon products (cracking) comprising contacting a hydrocarbon starting material with a catalytically active carbocatalyst to provide the corresponding one or more lower hydrocarbon products.

In one embodiment is a process comprising cracking and coupling. In embodiment is a process comprising cracking and dehydrogenation. In one embodiment is a process comprising metathesis and dehydrogenation. In one embodiment is a process comprising metathesis and coupling. In one embodiment is a process comprising metathesis and coupling and dehydrogenation.

In some embodiments, the carbocatalyst is selected from a fullerene-related material, amorphous carbon, crystalline carbon, mesoporous carbon, graphene oxide or graphite oxide derived material, or activated carbon.

In some embodiments, the carbocatalyst is selected from a fullerene-related material, amorphous carbon, crystalline carbon, graphene oxide or graphite oxide derived material, or activated carbon.

In some embodiments, the starting material in (a)-(d) is a material that does not comprise activated C—H bonds.

In some embodiments, the catalytically active carbocatalyst is a graphene oxide or graphite oxide derived material.

In some embodiments, the catalytically active carbocatalyst is characterized by one or more FT-IR features at about 3150 cm⁻¹, 1685 cm⁻¹, 1280 cm⁻¹, or 1140 cm⁻¹.

In some embodiments, the catalytically active carbocatalyst is a modified form of graphene oxide or graphite oxide (e.g., a further oxygenated form of graphene oxide or graphite oxide).

In some embodiments, the catalytically active carbocatalyst is a catalytically active surface-modified graphene oxide or graphite oxide.

In one embodiment of the process, the catalytically active surface-modified graphene oxide or graphite oxide is catalytically active surface-modified graphene oxide.

In one embodiment of the process, the catalytically active surface-modified graphene oxide or graphite oxide is catalytically active surface-modified graphite oxide.

In some embodiments, the catalytically active carbocatalyst has a surface modification comprising one or more of a hydrogen peroxide-terminated surface, an epoxide-terminated surface, a ketone-terminated surface, a diketone terminated surface, an aldehyde terminated surface, a carboxyl terminated surface, a hydroxyl terminated surface, an alcohol terminated surface, an ether terminated surface, a dioxirane terminated surface, a quinone terminated surface, a peroxy acid terminated surface, an ester terminated surface, an anhydride terminated surface or a perester terminated surface.

In some embodiments, the catalytically active carbocatalyst has a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, or an ether terminated surface.

In some embodiments, the catalytically active carbocatalyst has a surface modification comprising one or more of an epoxide-terminated surface, a ketone-terminated surface, an aldehyde terminated surface, a carboxyl terminated surface, a hydroxyl terminated surface, or an alcohol terminated surface.

In some embodiments, the catalytically active carbocatalyst has at least about 25% carbon and at least about 0.01% oxygen as measured by x-ray photoelectron spectroscopy (XPS). In alternative embodiments, the catalytically active carbocatalyst has at least about 25% carbon and at least about 0.01% oxygen as measured by elemental combustion analysis.

In some embodiments, the carbon-to-oxygen ratio for the catalytically active carbocatalyst is between about 1.5:1 and about 1:1.5 as measured by x-ray photoelectron spectroscopy (XPS).

In some embodiments, the carbon-to-oxygen ratio for the catalytically active carbocatalyst is between about 1:1 and about 5:1 as measured by x-ray photoelectron spectroscopy (XPS).

In some embodiments, the carbon-to-oxygen ratio for the catalytically active carbocatalyst is between about 1:1 and about 4:1 as measured by x-ray photoelectron spectroscopy (XPS).

In some embodiments, the carbon-to-oxygen ratio for the catalytically active carbocatalyst is between about 1:1 and about 3:1 as measured by x-ray photoelectron spectroscopy (XPS).

In some embodiments, the carbon-to-oxygen ratio for the catalytically active carbocatalyst is between about 2:1 and about 3:1 as measured by x-ray photoelectron spectroscopy (XPS).

For any of the embodiments described above, in some cases, provided herein is a process for converting
(a) a saturated hydrocarbon moiety on a hydrocarbon starting material to an unsaturated hydrocarbon moiety on a hydrocarbon product (dehydrogenation);
(b) a hydrocarbon starting material to one or more higher molecular weight hydrocarbon products (coupling);
(c) a hydrocarbon starting material to one or more metathesis products (metathesis);
(d) a higher hydrocarbon starting material to one or more lower hydrocarbon products (cracking);
or any combination thereof;
   under kinetic control comprising contacting any starting material in (a)-(d) with a catalytically active carbocatalyst to provide the corresponding product in (a)-(d).

In another aspect, provided herein is a process for converting ethane to ethene comprising contacting ethane with a catalytically active carbocatalyst to provide ethene.

In one embodiment, the carbocatalyst for the conversion of ethane to ethene is selected from a fullerene-related material, amorphous carbon, crystalline carbon, mesoporous carbon, graphene oxide or graphite oxide derived material, or activated carbon.

In another embodiment, the carbocatalyst for the conversion of ethane to ethene is selected from a fullerene-related material, amorphous carbon, crystalline carbon, graphene oxide or graphite oxide derived material, or activated carbon.

In some of the embodiments described above, the catalytically active carbocatalyst for the conversion of ethane to ethene is a graphene oxide or graphite oxide derived material.

In some of the embodiments described above, the catalytically active carbocatalyst for the conversion of ethane to ethene is characterized by one or more FT-IR features at about 3150 cm⁻¹, 1685 cm⁻¹, 1280 cm⁻¹, or 1140 cm⁻¹.

In some of the embodiments described above, the catalytically active carbocatalyst for the conversion of ethane to ethene is a catalytically active surface-modified graphene oxide or graphite oxide.

In one embodiment of the process, the catalytically active surface-modified graphene oxide or graphite oxide is catalytically active surface-modified graphene oxide.

In one embodiment of the process, the catalytically active surface-modified graphene oxide or graphite oxide is catalytically active surface-modified graphite oxide.

In some of the embodiments described above, the catalytically active carbocatalyst for the conversion of ethane to ethene has a surface modification comprising one or more of a hydrogen peroxide-terminated surface, an epoxide-terminated surface, a ketone-terminated surface, a diketone terminated surface, an aldehyde terminated surface, a carboxyl terminated surface, a hydroxyl terminated surface, an alcohol terminated surface, an ether terminated surface, a dioxirane terminated surface, a quinone terminated surface, a peroxy acid terminated surface, an ester terminated surface, an anhydride terminated surface or a perester terminated surface.

In some of the embodiments described above, the catalytically active carbocatalyst for the conversion of ethane to ethene has a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, or an ether terminated surface.

In some of the embodiments described above, the catalytically active carbocatalyst for the conversion of ethane to ethene has a surface modification comprising one or more of an epoxide-terminated surface, a ketone-terminated surface, an aldehyde terminated surface, a carboxyl terminated surface, a hydroxyl terminated surface, or an alcohol terminated surface.

In some of the embodiments described above, the catalytically active carbocatalyst for the conversion of ethane to ethene has at least about 25% carbon and at least about 0.01% oxygen as measured by x-ray photoelectron spectroscopy (XPS).

In some of the embodiments described above for the conversion of ethane to ethene, the carbon-to-oxygen ratio for the catalytically active carbocatalyst is between about 1.5:1 and about 1:1.5 as measured by x-ray photoelectron spectroscopy (XPS).

In some of the embodiments described above for the conversion of ethane to ethene, the carbon-to-oxygen ratio for the catalytically active carbocatalyst is between about 1:1 and about 5:1 as measured by x-ray photoelectron spectroscopy (XPS).

In some of the embodiments described above for the conversion of ethane to ethene, the carbon-to-oxygen ratio for the catalytically active carbocatalyst is between about 2:1 and about 3:1 as measured by x-ray photoelectron spectroscopy (XPS).

For any of the embodiments described above, in one case, provided herein is a process for converting ethane to ethene under kinetic control comprising contacting ethane with a catalytically active carbocatalyst to provide ethene.

For any embodiments described above or below, provided herein is a catalytically active carbocatalyst, wherein the catalytically active carbocatalyst is an oxidized form of graphite. For any embodiments described above or below, provided herein is a catalytically active carbocatalyst, wherein the catalytically active carbocatalyst is an oxidized form of graphene. In some embodiments, the catalytically active carbocatalyst is modified graphene oxide or graphite oxide. In certain embodiments, the catalytically active carbocatalyst is an oxidized carbon-containing material.

In certain embodiments, the catalytically active carbocatalyst is a heterogeneous catalyst. In some embodiments, the catalytically active carbocatalyst provides a reaction solution pH which is neutral upon dispersion in a reaction mixture. In certain embodiments, the catalytically active carbocatalyst provides a reaction solution pH which is acidic upon dispersion in a reaction mixture. In other embodiments, the catalytically active carbocatalyst provides a reaction solution pH which is basic upon dispersion in a reaction mixture. In some embodiments, the catalytically active carbocatalyst is present on a solid support. In certain embodiments, the catalytically active carbocatalyst is present within a solid support.

For any embodiments described above or below, provided herein are processes, wherein the process further comprises contacting the reactants with a co-catalyst. In some embodiments, the co-catalyst is an oxidation catalyst. In certain embodiments, the co-catalyst is a zeolite.

For any embodiments described above or below, provided herein are processes, where the process further comprises an additional oxidizing agent. In some embodiments of the methods and/or processes described above or below, the method comprises a solvent-free reaction. In certain embodiments of the method described above or below, the method comprises one or more gaseous reactants in contact with a carbocatalyst.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an illustrative example of a modified graphene oxide or graphite oxide (MG) that is used in the methods and Examples of the disclosure.

FIG. 2 shows an illustrative X-ray Photoelectron Spectroscopy (XPS) performed on samples of as-prepared graphene oxide or graphite oxide.

FIG. 3 shows an illustrative schematic of a generalized flow reactor coupled with an internal circulator that allows for unreacted hydrocarbon to be recovered and recirculated in order to achieve maximum yield over the course of multiple passes.

### DETAILED DESCRIPTION OF THE INVENTION

The manufacture of raw materials such as low molecular weight alkanes or alkenes has relied on cracking of naphtha. Naphtha is a component of natural gas condensate or a distillation product from petroleum, coal tar or peat. Naphtha is a mixture of hydrocarbons boiling in a certain temperature range covering certain low molecular weight and volatile fractions of liquid hydrocarbons in petroleum. For example, 95% of certain high volume hydrocarbons like low molecular weight alkenes produced at present are manufactured as a byproduct of cracking naphtha. However, the supply is being reduced and ethane crackers cannot produce alkenes as a recoverable byproduct. There is a need for methods of manufacturing high value commodities such as low molecular weight hydrocarbons while avoiding the industry's reliance on petroleum, which is a dwindling natural resource.

Provided herein are on-purpose methods for manufacture of high value chemicals using low cost starting materials and processes. Provided herein is practical technology for utilizing certain catalytically active carbocatalysts (e.g., surface-modified graphene and/or graphite oxide) for synthesis of low molecular weight saturated, partially saturated, or unsaturated organic compounds. In some cases, the compounds are gases and/or are volatile compounds. In some cases the compounds comprise one to six carbons. In some cases the compounds comprise one to five carbons. In some cases the compounds comprise one to four carbons. In some cases the compounds comprise one to three carbons. In some cases the compounds comprise two to six carbons. In some cases the compounds comprise two to five carbons. In some cases the compounds comprise two to four carbons. In some cases the compounds comprise two to three carbons. In some cases the compounds comprise three carbons.

There is a need for broad-spectrum catalysts that overcome one or more drawbacks of existing catalysts and that are able to catalyze a variety of chemical reactions (e.g., hydrocarbon transformations) using a wide range of initial reactants or starting materials.

Recognized herein are various limitations associated with current commercially-available methods for catalyzing hydrocarbon reactions. For instance, while transition metal-based catalysts may provide reactions rates that are commercially feasible, the use of metal catalysts has various drawbacks, such as metal contamination of the resulting products. This is particularly problematic in industries where the product is intended for health or biological use, or other uses sensitive to the presence of metals. The carbocatalysts described herein (e.g., graphene and/or graphite oxide) for hydrocarbon transformations (e.g., conversion of ethane to ethene) are substantially free of transition metals, which allows for production of pure hydrocarbons that are free of metal contaminants.

Another drawback of metal catalysts is that metal catalysts are typically not selective in hydrocarbon reactions. Transition metal based dehydrogenations of hydrocarbons are typically oxidative dehydrogenations (ODH) and require high ratios of oxygen/hydrocarbon, which leads to unwanted oxidation by-products. In some embodiments, provided herein are carbocatalysts (e.g., graphene oxide or graphite oxide) that allow for kinetic control of certain hydrocarbon reactions (e.g. dehydrogenation) which provides enhanced selectivity in hydrocarbon transformations.

As another example to illustrate the drawbacks of metal-based catalysts recognized herein, transition metal-based catalysts may be expensive to manufacture and processes employing such catalysts may have considerable startup and maintenance costs. By contrast, the carbon-based catalyst (e.g., graphene and/or graphite oxide) technologies described herein can be retrofitted to existing manufacturing facilities thus reducing any start up costs associated with ramping up new technologies for commercial large scale production of valuable hydrocarbons. Shale gas (including dry or wet shale gas) is abundant and can be easily transported to an existing manufacturing facility. Thus the versatile technologies described herein are not hindered by availability of starting materials and/or cost of procuring starting materials.

Described herein are processes for hydrocarbon transformations involving the use of carbocatalysts that combine the benefits of a metal-free synthesis along with the convenience of a heterogeneous work up. The methods provided herein are environmentally-friendly because they reduce the need for use of metal based catalysts in production plants. Further, the production steps are reduced, thereby reducing cost of goods and manufacturing services.

Typical hydrocarbon starting materials for hydrocarbon transformations often have sulfur containing impurities which poison metal based catalysts. An advantage of the catalytically active carbocatalysts (e.g., graphene and/or graphite oxide derived catalysts) described herein is that the catalysts are not poisoned by impurities (e.g., sulfur containing impurities) that typically poison other catalysts (e.g., transition metal based catalysts).

An additional advantage is that the catalytically active carbocatalysts (e.g., modified graphene and/or graphite oxide) described herein can be regenerated, thereby reducing industrial waste, and cost of goods and manufacturing. The carbocatalysts and processes utilizing such carbocatalysts that are described herein allow for activation of relatively inert starting materials such as hydrocarbons and are suitable for activation of C—H bonds and/or C—C bonds.

Certain previously used catalysts such as mesoporous carbon generate coke during the course of the reaction—a disadvantage which is overcome by the carbocatalysts (e.g., graphene and/or graphite oxide derived catalysts) described herein. Further, previously used catalysts have pores which delay the diffusion of products out of the mesoporous structures—a disadvantage which is overcome by the carbocatalysts (e.g., graphene and/or graphite oxide derived catalysts) described herein. Additionally, mesopoprous and/or amorphous carbon and/or activated carbon based catalysts clog reactors as a consequence of coking—a disadvantage which is overcome by the carbocatalysts (e.g., graphene and/or graphite oxide derived catalysts) described herein.

Certain previously described catalysts such as fullerene related materials or crystalline carbon materials have drawbacks. The introduction of potentially catalytically active edge sites or defect sites in fullerene-related materials or crystalline carbon, is challenging. By contrast, the catalytically active carbocatalysts provided herein (e.g., modified graphene oxide and/or graphite oxide) overcome the potential disadvantages of fullerene-related materials; in some cases the catalysts provided herein (e.g., graphene oxide and/or graphite oxide) have significantly higher per weight numbers of edge or corner sites compared to fullerene base materials. The catalytically active carbocatalysts provided herein (e.g., modified graphene oxide and/or graphite oxide) are powdered materials comprising a higher percentage of edge sites, compared to fullerene-related and/or crystalline carbon related materials.

Accordingly, the versatile carbocatalysts and processes utilizing such carbocatalysts that are described herein (e.g., graphene oxide and/or graphite oxide) are applicable to a variety of organic reactions including and not limited to hydrocarbon-hydrocarbon (e.g., C—C) couplings, alkane metathesis, alkane and/or cycloalkane cracking, and alkane and/or cycloalkane dehydrogenations. The carbocatalysts that are described herein (e.g., graphene oxide and/or graphite oxide) and comprise a higher concentration of reactive and/or selective sites on the surface and overcome the low overall activity and/or reactivity of previously described carbon-based catalysts.

The methods of the current disclosure also have applications in the pharmaceutical industry. For example, chalcones are important precursors for flavonoids and other pharmaceutically important materials and have many uses outside of the pharmaceutical industry. Additionally, the lack of metal in graphene oxide or graphite oxide allows for the use of these methods in reactions where metal contamination is a concern, such as reactions to produce pharmaceuticals or agricultural products, or in reactions where it would be detrimental, such as where the product will be subjected to further reactions or used in further applications that are sensitive to metal contamination.

### DEFINITIONS

The term “carbocatalyst,” as used herein, refers to a catalytically-active carbon containing material for the transformation or synthesis of various hydrocarbons as described herein. In one embodiment, the carbocatalysts described herein include graphite, graphite oxide, graphene, graphene oxide, partially oxidized graphite, partially oxidized graphene, or other related materials. In a further embodiment, the carbocatalysts described herein include surface-modified graphite, graphite oxide, graphene, graphene oxide, or other related materials. In further embodiments, the carbocatalysts described herein include graphite, graphite oxide, graphene, graphene oxide, partially oxidized graphite, partially oxidized graphene, fullerene related materials, amorphous carbon, crystalline carbon, activated carbon, carbon molecular sieves, or related materials.

In other embodiments, a carbocatalyst includes amorphous carbon, nanotubes, carbon molecular sieves, buckyballs, diamond, graphite fluoride, graphene fluoride, activated carbon, or other related materials, or a combination thereof. In some embodiments, a carbocatalyst includes amorphous carbon, crystalline carbon or fullerene related materials. In other embodiments, a carbocatalyst includes amorphous carbon, crystalline carbon, nanotubes, buckyballs, diamond, activated carbon, glassy carbon, charcoal, activated charcoal and intermediates or various combinations or mixtures of these carbon forms. In further embodiments, a carbocatalyst includes porous carbons, mesoporous carbons, microporous carbons, partially oxidized porous carbon, partially oxidized mesporous carbon, or partially oxidized microporous carbon. In further embodiments, a carbocatalyst comprises carbon composites.

As used herein fullerene-related material includes, for example, fullerenes, nanotubes, nanospheres, nanocones, nanofolders, nanobundles, onion-like carbons and the like. As used herein, amorphous carbon refers to an allotrope of carbon that does not have any crystalline structure. Amorphous carbon also includes glassy carbon. As used herein, crystalline carbon includes, for example, diamond, nanodiamond and the like. As used herein, mesoporous carbon comprises material with pores diameters between 2 and 50 nm. Microporous carbon comprises material with pore diameters of less than 2 nm. Activated carbon refers to material which is highly porous.

The term “catalytically active carbocatalyst,” as used herein, refers to a carbocatalyst substance or species that facilitates one or more chemical reactions. The term “catalytically active carbocatalyst” refers to a carbocatalyst which is catalytically active. A catalytically active carbocatalyst includes one or more reactive active sites for facilitating a chemical reaction, such as, for example, surface moieties (e.g., OH groups, epoxides, aldehydes, carboxylic acids or any other group described herein). The term catalytically active carbocatalyst includes a surface-modified graphene oxide, graphite oxide, or other carbon and oxygen-containing material that facilitates a chemical reaction, such as a hydrocarbon-hydrocarbon C—C coupling, a hydrocarbon cracking and/or metathesis and/or a dehydrogenation reaction.

The term “spent catalyst” or “spent carbocatalyst,” as used herein, refers to a catalyst that has been exposed to a reactant to generate a product. In some situations, a spent catalyst is incapable of facilitating a chemical reaction. A spent catalyst has reduced activity with respect to a freshly generated catalyst (also “fresh catalyst” herein). The spent catalyst is partially or wholly deactivated. In some cases, such reduced activity is ascribed to a decrease in the number of reactive active sites. A spent carbocatalyst is optionally regenerated as described herein to provide a catalytically active carbocatalyst.

The term “heterogeneous catalyst” or “heterogeneous carbocatalyst,” as used herein, refers to a solid-phase species configured to facilitate a chemical transformation. In heterogeneous catalysis, the phase of the heterogeneous catalyst generally differs from the phase of the reactants(s). A heterogeneous catalyst includes a catalytically active material on a solid support. In some cases the support is catalytically active or inactive. In some situations, the catalytically active material and the solid support is collectively referred to as a “heterogeneous catalyst” (or “catalyst”).

The term “solid support,” as used herein, refers to a support structure for holding or supporting a catalytically active material, such as a catalyst (e.g., carbocatalyst). In some cases, a solid support does not facilitate a chemical reaction. However, in other cases the solid support takes part in a chemical reaction.

The term “nascent catalyst” or “nascent carbocatalyst,” as used herein, refers to a substance or material that is used to form a catalyst. A nascent catalyst is characterized as a species that has the potential for acting as a catalyst, such as, upon additional processing or chemical and/or physical (e.g., surface) modification or transformation.

As used herein, the “carbon-to-oxygen ratio” for the catalytically active carbocatalyst described herein (e.g., surface-modified graphene oxide or graphite oxide) refers to the relative amounts of carbon and oxygen in the catalyst. Thus, by way of example, a carbon-to oxygen ratio of 1 to 1 refers to a catalyst composition comprising equal amounts of carbon and oxygen. It will be understood that the catalyst also comprises hydrogen.

Accordingly, in some embodiments, a catalytically active carbocatalyst described herein (e.g., surface-modified graphene oxide or graphite oxide) has a carbon-to-oxygen ratio of between about 1 to 1.5 to about 1.5 to 1. In other embodiments, a catalytically active carbocatalyst described herein (e.g., surface-modified graphene oxide or graphite oxide) has a carbon-to-oxygen ratio of between about 1 to 1 to about 9 to 1. In yet other embodiments, a catalytically active carbocatalyst described herein (e.g., surface-modified graphene oxide or graphite oxide) has a carbon-to-oxygen ratio of between about 1 to 1 to about 5 to 1, about 1 to 1 to 4 to 1, about 1 to 1 to 3 to 1 or about 1 to 1 to 2 to 1, or about 2 to 1 to about 3 to 1.

The term “surface,” as used herein, refers to the boundary between a liquid and a solid, a gas and a solid, a solid and a solid, or a liquid and a gas. A species on a surface has decreased degrees of freedom with respect to the species in the liquid, solid or gas phase.

A “surface-modified graphite oxide or graphene oxide” refers to graphite or graphene oxide which is terminated with certain functional groups. By way of example, a surface-modified graphite oxide or graphene oxide having a “hydrogen peroxide terminated surface” refers to the attachment of an —R—OOH or —ROO⁻ moiety to the graphene oxide or graphite oxide surface, where R is bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like.

A “peroxide terminated surface” refers to the attachment of an —R—OOR′ moiety to the graphene oxide or graphite oxide surface, where R is bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, or any suitable monovalent group such as an alkyl, an aryl, a cycloalkyl, an alkene, an alkyne, a heteroaryl, a cycloalkene, a heterocycle, a heterocycloalkene, and the like.

“Anhydride terminated surface” refers to the attachment of an —R—C(═O)O—C(═O)—R′ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cyclic anhydride attached to the surface.

“Carbonate terminated surface” refers to the attachment of an —R—OC(═O)O—R′ or —R—OC(═O)O⁻ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cyclic carbonate attached to the surface.

“Aldehyde terminated surface” refers to the attachment of an —R—C(═O)H moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like.

“Ketone terminated surface” refers to the attachment of an —R—C(═O)—R′ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cyclic ketone attached to the surface.

“Epoxide terminated surface” refers to the attachment of a

[chemical structure]

moiety to the graphene oxide or graphite oxide surface, where R is bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, H, or any suitable monovalent group such as an alkyl, an aryl, a cycloalkyl, an alkene, an alkyne, a heteroaryl, a cycloalkene, a heterocycle, a heterocycloalkene, and the like.

“Carboxyl terminated surface” refers to the attachment of an —R—C(═O)—OH or —R—C(═O)O⁻ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like.

“Ester terminated surface” refers to the attachment of an —R—C(═O)—OR′ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cyclic ester attached to the surface.

“Peroxy acid terminated surface” refers to the attachment of an —R—C(═O)—OOH or —R—C(═O)OO⁻ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like.

“Perester terminated surface” refers to the attachment of an —R—C(═O)−OOR′ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cyclic perester attached to the surface.

“Diketone terminated surface” refers to the attachment of an —R—C(═O)—C(═O)—R′ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, H, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cyclic diketone attached to the surface.

“Alcohol terminated surface” refers to the attachment of an —R—OH moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like. “Hydroxyl terminated surface” refers to the attachment of a —OH moiety to the graphene oxide or graphite oxide surface.

“Ether terminated surface” refers to the attachment of a

[chemical structure]

moiety to the graphene oxide or graphite oxide surface, where R is bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cyclic ether attached to the surface.

“Dioxirane terminated surface” refers to the attachment of a

[chemical structure]

moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, H, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a cycle, bearing an dioxirane, attached to the surface.

“Quinone terminated surface” refers to the attachment of a

[chemical structure]

para quinone moiety or a

[chemical structure]

ortho quinone moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and R′ is R attached to the surface, H, or any suitable monovalent group such as an alkyl, an aryl, a cycloalkyl, an alkene, an alkyne, a heteroaryl, a cycloalkene, a heterocycle, a heterocycloalkene, and the like, or R and R′ together form a fused cycle attached to the surface.

“Nitro terminated surface” refers to the attachment of an —R—NO₂ moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like.

“Nitroso terminated surface” refers to the attachment of an —R—NO moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like.

“Iodo terminated surface”, “Bromo terminated surface”, “Fluoro terminated surface” and “chloro terminated surface” refers to attachment of a —R—X moiety to the graphene oxide or graphite oxide surface, where R is a bond or any suitable bivalent group such as an alkylene, an arylene, a cycloalkylene, an alkenylene, an alkynylene, a heteroarylene, a cycloalkenylene, a heterocycloalkylene, a heterocycloalkenylene and the like, and X is F, Cl, Br, or I.

“Alkane” means a linear saturated hydrocarbon of one to 50 carbon atoms or a branched saturated hydrocarbon radical of three to 50 carbon atoms, e.g., methane, ethane, propane, butane (including all isomeric forms), pentane (including all isomeric forms), and the like.

“Cycloalkyl” or “cycloalkane” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like. In some embodiments a cycloalkyl group is covalently bonded to a surface as described herein.

“Hydrocarbon” means a compound or moiety comprising carbon-carbon bonds and carbon-hydrogen bonds. The carbon-carbon bonds in a hydrocarbon may be saturated C—C bonds, or —C═C— bonds or —C≡C— bonds, or any combination thereof.

“Activated C—H bond” refers to C—H bonds that are in the vicinity of groups that render the hydrogen easily abstractable. By way of example, an activated C—H bond is present at the benzylic position of an ethyl benzene.

### Carbocatalysts

The ability of various carbon-based materials to catalyze the extremely wide number of possible chemical reactions has hitherto not been explored in detail. To date such efforts have relied on exploitation of the relatively high surface areas intrinsic to carbon-based materials to enhance the activity of transition metal based catalysts. For example, metal catalysts have been placed on graphene-based materials to take advantage of the high surface area of such materials and to enhance the activities of the transition metal-based catalysts. Although non-carbon materials (e.g., metals, metal oxides, non-metals and non-metal oxides) have been placed on carbon or derivatized carbon surfaces (e.g., graphene oxide materials) to form supported catalysts, the catalytic activity is attributable to non-carbon material and the carbon or derivatized carbon surface serves merely to support the metal catalyst. In contrast, the carbocatalysts described herein are catalytically active by themselves. When additional catalytically-active materials are present (e.g., optional solid acid catalysts), the carbocatalyst continues to be catalytically active and the resulting material can be characterized as a catalyst mixture of a carbocatalyst and a solid acid catalyst.

Recognized herein is the importance of the role of catalyst morphology in hydrocarbon transformations. The carbocatalysts described herein (e.g., graphite or graphene oxide) comprise accessible edges and/or corners which anchor reactive surface groups and/or provide a higher surface concentration of reactive species.

The carbocatalysts (e.g., graphene and/or graphite oxide), and processes involving the use of said carbocatalysts, which are described herein are useful for the synthesis of a large number of industrially and commercially important chemicals that would otherwise be difficult or prohibitively expensive to produce. Additionally, some useful chemical reactions involving organic materials have no available catalysts and are therefore unduly slow or costly. In some embodiments, the carbocatalysts provided herein (e.g., graphene and/or graphite oxide) provide access to such previously intractable chemistries such as hydrocarbon-hydrocarbon coupling, hydrocarbon dehydrogenation and/or metathesis and/or cracking. The broad-spectrum catalysts described herein are able to catalyze a variety of chemical reactions using a variety of initial products (starting materials) and provide a non-toxic alternative to other catalysts and/or reactions. The broad spectrum catalyst and methods of using such catalysts that are provided herein overcome one or more drawbacks of existing catalysts and/or processes.

Methods of Preparing Catalytically Active Carbocatalysts

In one aspect, a carbocatalyst suitable for hydrocarbon transformations described herein (e.g., conversion of ethane to ethene) is an oxidized form of graphite, e.g., a graphene oxide or graphite oxide based catalyst. Graphene oxide or graphite oxide used as a catalyst in the present disclosure is produced using known methods. For example, modified graphene oxide or graphite oxide is produced by the oxidation of graphite using KMnO₄ and NaNO₃ in concentrated sulfuric acid in concentrated sulfuric acid as described in W. S. Hummer Jr. R. E. Offeman, J. Am. Chem. Soc. 80: 1339 (1958) and A. Lerf, et al. J. Phys Chem. B 102: 4477-4482 (1998), both incorporated in material part by reference herein. Graphene oxide or graphite oxide may also be produced by the oxidation of graphite using NaClO₃ in H₂SO₄ and fuming HNO₃ as described in L. Staudenmaier, Ber. Dtsch. Chem. Ges. 31: 1481-1487 (1898); L. Stuadenmaier, Ber. Dtsch. Chem. Ges. 32:1394-1399 (1899); T. Nakajima, et al. Carbon 44: 537-538 (2006), all incorporated in material part by reference herein. Graphene oxide or graphite oxide may also be prepared by a Brodie reaction.

Where the use of large amounts of strong oxidants and acids (e.g., KMnO₄ and H₂SO₄) is not desirable for large scale commercialization, contemplated herein is the use of milder and readily available oxidants such as hydrogen peroxide and Oxone (potassium peroxymonosulfate; KHSO₅) to be used in lieu of KMnO₄. Optionally carbon materials can be oxygenated through pyrolysis, a process which uses ambient molecular oxygen (O₂) to oxidize a carbon surface.

In some embodiments, a method for forming a catalytically-active graphene oxide or catalytically-active graphite oxide catalyst from a nascent catalyst comprises providing the nascent catalyst to a reaction chamber (or “reaction vessel”), the nascent catalyst comprising graphene or graphite on a solid support. Next, the nascent catalyst is heated in the reaction chamber to an elevated temperature. The nascent catalyst is then contacted with a chemical oxidant.

In some embodiments, the chemical oxidant includes at least one or more materials selected from the group consisting of potassium permanganate, hydrogen peroxide, organic peroxides, peroxy acids, ruthenium-containing species (e.g., tetrapropylammonium perruthenate or other perruthenates), lead-containing species (e.g., lead tetraacetate), chromium-containing species (e.g., chromium oxides or chromic acids), iodine-containing species (e.g., periodates), sulfur-containing oxidants (e.g., potassium peroxymonosulfate or sulfur dioxide), molecular oxygen, ozone, chlorine-containing species (e.g., chlorates or perchlorates or hypochlorites), sodium perborate, nitrogen-containing species (e.g., nitrous oxide or dinitrogen tetraoxide), silver containing species (e.g., silver oxide), osmium containing species (e.g., osmium tetraoxide), 2,2′-dipyridyldisulfide, cerium-containing species (e.g., ammonium cerium nitrate), benzoquinone, Dess Martin periodinane, meta-chloroperbenzoic acid, molybdenum containing species (e.g., molybdenum oxides), N-oxides (e.g., pyridine N-oxide), vanadium-containing species (e.g., vanadium oxides), (2,2,6,6-tetramethylpiperidin-1-yl)oxidanyl (TEMPO), or iron-containing species (e.g., potassium ferricyanide).

In other embodiments, the chemical oxidant is a plasma excited species of an oxygen-containing chemical. In an example, the chemical oxidant includes plasma-excited species of O₂, H₂O₂, NO, NO₂, or other chemical oxidants. In such a case, the nascent catalyst in the reaction chamber is contacted with plasma excited species of the oxygen-containing chemical continuously, such as for a predetermined period of time of at least about 0.01 seconds, or 0.1 seconds, or 1 second, or 10 seconds, or 30 seconds, or 1 minute, or 5 minutes, or 10 minutes, or 15 minutes, or 20 minutes, or 30 minutes, or 1 hour, or 2 hours, or 3 hours, or 4 hours, or 5 hours, or 6 hours, or 12 hours, or 1 day, or 2 days, or 3 days, or 4 days, or 5 days, or 6 days, or 1 week, or 2 weeks, or 3 weeks, or 1 month, or 2 months, or 3 months, or 4 months, or 5 months, or 6 months. Alternatively, the nascent catalyst in the reaction chamber is contacted with plasma excites species of the oxygen-containing chemical in pulses, such as pulses having a duration of at least about 0.1 seconds, or 1 second, or 10 seconds, or 30 seconds, or 1 minute, or 10 minutes, or 30 minutes, or 1 hour, or 2 hours, or 3 hours, or 4 hours, or 5 hours, or 6 hours, or 12 hours, or 1 day, or 2 days, or 3 days, or 4 days, or 5 days, or 6 days, or 1 week, or 2 weeks, or 3 weeks, or 1 month, or 2 months, or 3 months, or 4 months, or 5 months, or 6 months. In some situations, the nascent catalyst is exposed to the chemical oxidant for a time period between about 0.1 seconds and 100 days.

In some situations, the nascent catalyst is heated during exposure to the chemical oxidant. In an example, the nascent catalyst is heated at a temperature between about 20° C. and 3000° C., or 20° C. and 2000° C., or about 100° C. and 2000° C.

Alternatively, a method for forming a catalytically-active graphene oxide or catalytically-active graphite oxide catalyst from a nascent catalyst includes providing a nascent catalyst comprising graphene or graphite to a reaction chamber. The reaction chamber has a holder or susceptor for holding one or more nascent catalysts. Next, the nascent catalyst is contacted with one or more acids. In some cases, the one or more acids include sulfuric acid. In some cases, the nascent catalyst is pretreated with potassium persulfate before contacting the nascent catalyst with the one or more acids. Next, the nascent catalyst is contacted with a chemical oxidant. Next, the nascent catalyst is contacted with hydrogen peroxide.

As another alternative, a method for forming a catalytically-active graphene oxide or catalytically-active graphite oxide catalyst from a nascent catalyst includes providing a nascent catalyst comprising graphene or graphite to a reaction chamber. Next, the nascent catalyst is contacted with one or more acids. In some cases, the nascent catalyst is pretreated with potassium persulfate before the nascent catalyst is contacted with the one or more acids. In some cases, the one or more acids include sulfuric acid and nitric acid. The nascent catalyst is then contacted with sodium chlorate, potassium chlorate and/or potassium perchlorate.

In some embodiments, a method for forming a catalytically active carbocatalyst comprises providing a carbon-containing material in a reaction chamber and contacting the carbon-containing material in the reaction chamber with an oxidizing chemical (also “chemical oxidant” herein) for a predetermined period of time until the carbon-to-oxygen ratio of the carbon-containing material is less than or equal to about 1,000,000 to 1. In some cases, the ratio is determined via elemental analysis, such as XPS. In some embodiments, the time sufficient to achieve such carbon-to-oxygen ratio is at least about 0.1 seconds, or 1 second, or 10 seconds, or 30 seconds, or 1 minute, or 10 minutes, or 30 minutes, or 1 hour, or 2 hours, or 3 hours, or 4 hours, or 5 hours, or 6 hours, or 12 hours, or 1 day, or 2 days, or 3 days, or 4 days, or 5 days, or 6 days, or 1 week, or 2 weeks, or 3 weeks, or 1 month, or 2 months, or 3 months, or 4 months, or 5 months, or 6 months. In some cases, the carbon-containing material is contacted with the chemical oxidant until the carbon-to-oxygen ratio, as determined by elemental analysis, is less than or equal to about 500,000 to 1, or 100,000 to 1, or 50,000 to 1, or 10,000 to 1, or 5,000 to 1, or 1,000 to 1, or 500 to 1, or 100 to 1, or 50 to 1, or 10 to 1, or 5 to 1, or 1 to 1.

As an alternative, a method for forming oxidized and catalytically-active graphite or oxidized and catalytically-active graphene comprises providing graphite or graphene in a reaction chamber and contacting the graphite or graphene with an oxidizing chemical until an infrared spectroscopy spectrum of the graphite or graphene exhibits one or more FT-IR features at about 3150 cm⁻¹, 1685 cm⁻¹, 1280 cm⁻¹, or 1140 cm⁻¹. In such embodiments, the catalytically active carbocatalyst having one or more FT-IR features at about 3150 cm⁻¹, 1685 cm⁻¹, 1280 cm⁻¹, or 1140 cm⁻¹ comprises surface-modified graphene oxide or graphite oxide.

Regeneration of Spent Catalyst

In some embodiments, methods for regenerating a spent catalyst, such as a catalytically active carbocatalyst described herein (e.g., graphene and/or graphite oxide) (e.g., graphene and/or graphite oxide), include providing the spent catalyst in a reaction chamber or vessel and contacting the spent catalyst with a chemical oxidant. In some cases, the chemical oxidant includes one or more material selected from the group above. In other cases, the chemical oxidant is a plasma excited species of an oxygen-containing chemical. In an example, the chemical oxidant includes plasma-excited species of O₂, H₂O₂, NO, NO₂, or other chemical oxidants. In some embodiments, the spent catalyst is contacted with the chemical oxidant continuously or in pulses, as described above. Contacting the spent catalyst with the chemical oxidant produces a carbocatalyst having a catalytically active material. In an example, contacting a spent catalyst covered with graphene or graphite (or other carbon-containing and oxygen deficient material) forms a layer of catalytically-active graphene oxide or graphite oxide.

Also contemplated with the scope of the present disclosure are other methods of preparation of catalytically active graphene oxide or graphite oxide as described in PCT International Application PCT/US2011/38327 (WO 2011/150325) which disclosure is incorporated herein by reference. Also contemplated within the scope of the present disclosure are variations of catalytically active graphene oxide or graphite oxide, including variations in island shapes, coverage and/or adsorption locations, as described in co-pending PCT International Application PCT/US2011/38334 (WO 2011/150329) which disclosure is incorporated herein by reference.

An advantage of catalytically active graphene oxide or graphite oxide catalyzed hydrocarbon transformations described herein (e.g., conversion of ethane to ethene) is that the carbocatalyst is heterogeneous, i.e. it does not dissolve in the reaction mixture. Many starting materials, such as alcohols, aldehydes, alkynes, methyl ketones, olefins, methyl benzenes, thiols, and disubstituted methylenes, and their reaction products are soluble in a wide range of organic solvents. In chemical reactions comprising such dissolved starting materials, the graphene oxide or graphite oxide remains as a suspended solid throughout the chemical reaction. In some of the aforementioned methods, the graphene oxide or graphite oxide is removed from the reaction product using simple mechanical methods, such as filtration, centrifugation, sedimentation, or other appropriate mechanical separation techniques, eliminating the need for more complicated techniques such as chromatography or distillation to remove the catalyst.

Following a catalytic reaction, the graphene oxide or graphite oxide is in a different chemical form or in the same chemical form. For example, in one embodiment, hydrocarbon transformations described herein (e.g., conversion of ethane to ethene) result in slow reduction or deoxygenation of the graphene oxide or graphite oxide and loss of functional groups. This altered graphene oxide or graphite oxide remaining after catalysis is put to other uses, or it is regenerated. For example, following the catalytic reaction, the graphene oxide or graphite oxide is in a reduced form. This material is very similar to graphene or graphite and may simply be used for graphene or graphite purposes. For example, reduced graphene oxide is used in energy storage devices or field effect transistors. Alternatively, the reduced graphene oxide or graphite oxide is reoxidized to regenerate the graphene oxide or graphite oxide catalyst. In a further embodiment, following a reaction, graphene oxide or graphite oxide used in the reaction is regenerated in situ and is in the same form as at the start of the reaction. Reoxidation methods are the same as those used to generate the graphene oxide or graphite oxide catalyst originally, such as a Hummers, Staudenmaier, or Brodie oxidation. Thus the carbocatalysts described herein provide an economical alternative to metal based catalysts.

In some embodiments of the invention, carbocatalysts are described that are configured for use with cracking, metathesis, dehydrogenation and/or hydrocarbon coupling reactions. In some embodiments of the invention, carbocatalysts are described that are configured for C—H bond activation in reactions involving hydrocarbons. Such carbocatalysts enable reaction rates up to and even exceeding that of transition metal-based catalysts, but reduce, if not eliminate, the contamination issues associated with the use of transition metal-based catalysts.

In one embodiment, a carbocatalyst used as a catalyst for any transformation described herein is catalytically active graphene oxide or graphite oxide which comprises one or more oxygen-containing functionalities. An example graphene oxide or graphite oxide catalyst is shown in FIG. 1. In specific embodiments, a graphene oxide or graphite oxide based carbocatalyst described herein (e.g., graphene and/or graphite oxide) (e.g., graphene and/or graphite oxide) contains surface moieties comprise of one or more of hydroxyls, alcohols, epoxides, ketones, aldehydes or carboxylic acids. In some situations, at least some of the oxygen-containing functional groups is used to oxidize organic species, such as alkanes or cycloalkanes. In other cases, oxygen is used as a terminal oxidant. Various embodiments of the invention describe carbocatalysts having graphene oxide at various compositions, concentrations and islands shapes, coverage and adsorption locations.

Carbon-containing catalysts provided herein include unsupported catalytically-active graphene or catalytically-active graphite oxide, as well as graphene oxide or graphite oxide on a solid support, such as a carbon-containing solid support or metal-containing solid support (e.g., TiO₂, Al₂O₃). In alternate embodiments, a solid support is a polymer with a catalytically active graphite oxide or graphene oxide dispersed in the polymer. In some embodiments, catalysts are provided having catalytically-active graphene oxide and/or catalytically-active graphite oxide on a solid support. Examples of such solid supports include carbon nitride, boron nitride, boron-carbon nitride and the like. In other embodiments, catalysts are provided having a catalytically-active carbon and oxygen-containing material and a co-catalyst such as carbon nitride, boron nitride, boron-carbon nitride and the like.

In further embodiments, carbon-containing catalysts provided herein include unsupported catalytically-active graphene or catalytically-active graphite oxide, as well as graphene oxide or graphite oxide within a solid support, such as a zeolite, a polymer and/or metal-containing solid support (e.g., TiO₂, Al₂O₃). In some embodiments, catalysts are provided having catalytically-active graphene oxide and/or catalytically-active graphite oxide within a polymer support. In further embodiments, catalysts are provided having catalytically-active graphene oxide and/or catalytically-active graphite oxide within an amorphous solid, e.g., activated charcoal, coal fly ash, bio ash or pumice. In other embodiments, catalysts are provided having a catalytically-active carbon and oxygen-containing material and a co-catalyst such as carbon nitride, boron nitride, boron-carbon nitride and the like.

Metal Content

In some embodiments, a heterogeneous catalytically-active graphene oxide or graphite oxide catalyst (or other carbon and oxygen-containing catalyst, or a carbocatalyst) is substantially free of metal, particularly transition metal. In some embodiments, a heterogeneous catalytically-active surface-modified graphene oxide or graphite oxide catalyst (or other carbon and oxygen-containing catalyst, or a carbocatalyst) suitable for reactions described herein comprises less than 5% of a transition metal by weight of the catalyst. In some embodiments, a heterogeneous catalytically-active surface-modified graphene oxide or graphite oxide catalyst (or other carbon and oxygen-containing catalyst, or a carbocatalyst) suitable for reactions described herein comprises less than 2% of a transition metal by weight of the catalyst. In some embodiments, a heterogeneous catalytically-active surface-modified graphene oxide or graphite oxide catalyst (or other carbon and oxygen-containing catalyst, or a carbocatalyst) suitable for reactions described herein comprises less than 1% of a transition metal by weight of the catalyst. In some embodiments, a heterogeneous catalytically-active surface-modified graphene oxide or graphite oxide catalyst (or other carbon and oxygen-containing catalyst, or a carbocatalyst) suitable for reactions described herein comprises less than 0.5% of a transition metal by weight of the catalyst. In some embodiments, a heterogeneous catalytically-active surface-modified graphene oxide or graphite oxide catalyst (or other carbon and oxygen-containing catalyst, or a carbocatalyst) suitable for reactions described herein comprises less than 0.1% of a transition metal by weight of the catalyst. In some embodiments, a heterogeneous catalytically-active surface-modified graphene oxide or graphite oxide catalyst (or other carbon and oxygen-containing catalyst, or a carbocatalyst) suitable for reactions described herein comprises less than 0.01% of a transition metal by weight of the catalyst. In some embodiments, a heterogeneous catalytically-active surface-modified graphene oxide or graphite oxide catalyst (or other carbon and oxygen-containing catalyst, or a carbocatalyst) suitable for reactions described herein comprises less than 0.001% of a transition metal by weight of the catalyst.

In some cases, the heterogeneous catalyst has a substantially low metal (e.g., transition metal) concentration of metals selected from the group consisting of W, Fe, Ta, Ni, Au, Ag, Rh, Ru, Pd, Pt, Ir, Co, Mn, Os, Zr, Zn, Mo, Re, Cu, Cr, V, Ti and Nb. In an embodiment, the heterogeneous catalyst has a transition metal concentration that is less than or equal to about 50 part per million, about 20 part per million, about 10 part per million, about 5 part per million, about 1 part per million (“ppm”), or 0.5 ppm, or 0.1 ppm, or 0.06 ppm, or 0.01 ppm, or 0.001 ppm, or 0.0001 ppm, or 0.00001 ppm as measured by atomic absorption spectroscopy or mass spectrometry (e.g., inductively coupled plasma mass spectrometry, or “ICP-MS”). In another embodiment, the heterogeneous catalyst has a metal content (mole %) that is less than about 0.0001%, or less than about 0.000001%, or less than about 0.0000001%.

In some cases, a heterogeneous catalytically-active graphene oxide or graphite oxide catalyst (or other carbon and oxygen-containing catalyst) has a substantially low manganese content. In one example the particles have a manganese content that is less than about 1 ppm, or 0.5 ppm, or 0.1 ppm, or 0.06 ppm, or 0.01 ppm, or 0.001 ppm, or 0.0001 ppm, or 0.00001 ppm as measured by atomic absorption spectroscopy or mass spectrometry (e.g., inductively coupled plasma mass spectrometry, or “ICP-MS”).

In some situations, catalysts provided herein have a certain level of transition metal content. As an example, a carbocatalyst suitable for any reaction described herein includes graphene oxide or graphite oxide and has a transition metal content between about 1 part per million and about 50% by weight of the catalyst. In some cases, the transition metal content of the carbocatalyst is between about 1 part per million and about 25% by weight of the catalyst, or between about 1 part per million and about 10% by weight of the catalyst, or between about 1 part per million and about 5% by weight of the catalyst, or between about 1 part per million and about 1% by weight of the catalyst, or between about 10 part per million and about 50% by weight of the catalyst, or between about 100 part per million and about 50% by weight of the catalyst, or between about 1000 part per million and about 50% by weight of the catalyst, or between about 10 part per million and about 25% by weight of the catalyst, or between about 100 part per million and about 25% by weight of the catalyst, or between about 1000 part per million and about 25% by weight of the catalyst, or between about 10 part per million and about 10% by weight of the catalyst, or between about 100 part per million and about 10% by weight of the catalyst, or between about 1000 part per million and about 10% by weight of the catalyst, or between about 10 part per million and about 5% by weight of the catalyst, or between about 100 part per million and about 5% by weight of the catalyst, or between about 1000 part per million and about 5% by weight of the catalyst, or between about 10 part per million and about 1% by weight of the catalyst, or between about 100 part per million and about 1% by weight of the catalyst, or between about 1000 part per million and about 1% by weight of the catalyst.

In some situations, the transition metal content of the carbocatalyst is determined by atomic absorption spectroscopy (AAS) or other elemental analysis technique, such as x-ray photoelectron spectroscopy (XPS), or mass spectrometry (e.g., inductively coupled plasma mass spectrometry, or “ICP-MS”).

In some embodiments, the carbocatalyst has a low concentration of transition metals selected from the group consisting of W, Fe, Ta, Ni, Au, Ag, Rh, Ru, Pd, Pt, Ir, Co, Mn, Os, Zr, Zn, Mo, Re, Cu, Cr, V, Ti and Nb. In some embodiments, a carbocatalyst has a metal content (mole %) that is more than about 0.0001%, and up to about 50 mole % of the total weight of the catalyst, or more than about 0.001%, and up to about 50 mole % of the total weight of the catalyst, more than about 0.01%, and up to about 50 mole % of the total weight of the catalyst, more than about 0.1%, and up to about 50 mole % of the total weight of the catalyst, more than about 0.0001%, and up to about 25 mole % of the total weight of the catalyst, or more than about 0.001%, and up to about 25 mole % of the total weight of the catalyst, more than about 0.01%, and up to about 25 mole % of the total weight of the catalyst, more than about 0.1%, and up to about 25 mole % of the total weight of the catalyst, more than about 0.0001%, and up to about 10 mole % of the total weight of the catalyst, or more than about 0.001%, and up to about 10 mole % of the total weight of the catalyst, more than about 0.01%, and up to about 10 mole % of the total weight of the catalyst, more than about 0.1%, and up to about 10 mole % of the total weight of the catalyst, more than about 0.0001%, and up to about 5 mole % of the total weight of the catalyst, or more than about 0.001%, and up to about 5 mole % of the total weight of the catalyst, more than about 0.01%, and up to about 5 mole % of the total weight of the catalyst, more than about 0.1%, and up to about 5 mole % of the total weight of the catalyst. more than about 0.0001%, and up to about 1 mole % of the total weight of the catalyst, or more than about 0.001%, and up to about 1 mole % of the total weight of the catalyst, more than about 0.01%, and up to about 1 mole % of the total weight of the catalyst, more than about 0.1%, and up to about 1 mole % of the total weight of the catalyst.

Surface

In some embodiments, a non-transition metal catalyst having catalytically-active graphene oxide or graphite oxide has a surface that is configured to come in contact with a reactant, such as a hydrocarbon for dehydrogenation, metathesis, cracking or coupling. In some cases, the catalyst has a surface that is terminated by one or more of hydrogen peroxide, hydroxyl groups (OH), epoxide groups, ketone groups, aldehyde groups, or carboxylic acid groups. In some embodiments, a catalytically active carbocatalyst (e.g., graphene oxide or graphite oxide) comprises surface groups rich in electrons which coordinate a redox process. In some embodiments, a catalytically active carbocatalyst (e.g., graphene oxide or graphite oxide) comprises Lewis basic sites which can abstract hydrogen from C—H bonds of hydrocarbons (e.g., ethane or butane) to produce dehydrogenated products (e.g., ethene, butene).

In an embodiment, the catalyst has a surface that includes one or more species (or “surface moieties”) selected from the group consisting of hydroxyl group, alkyl group, aryl group, alkenyl group, alkynyl group, epoxide group, peroxide group, peroxyacid group, aldehyde group, ketone group, ether group, carboxylic acid or carboxylate group, peroxide or hydroperoxide group, lactone group, thiolactone, lactam, thiolactam, quinone group, anhydride group, ester group, carbonate group, acetal group, hemiacetal group, ketal group, hemiketal group, amino, hydroxyamino, aminal, hemiaminal, carbamate, isocyanate, isothiocyanate, cyanamide, hydrazine, hydrazide, carbodiimide, oxime, oxime ether, N-heterocycle, N-oxide, hydroxylamine, hydrazine, semicarbazone, thiosemicarbazone, urea, isourea, thiourea, isothiourea, enamine, enol ether, aliphatic, aromatic, phenolic, thiol, thioether, thioester, dithioester, disulfide, sulfoxide, sulfone, sultone, sulfinic acid, sulfenic acid, sulfenic ester, sulfonic acid, sulfite, sulfate, sulfonate, sulfonamide, sulfonyl halide, thiocyanate, thiol, thial, S-heterocycle, silyl, trimethylsilyl, phosphine, phosphate, phosphoric acid amide, thiophosphate, thiophosphoric acid amide, phosphonate, phosphinite, phosphite, phosphate ester, phosphonate diester, phosphine oxide, amine, imine, amide, aliphatic amide, aromatic amide, halogen, chloro, iodo, fluoro, bromo, acyl halide, acyl fluoride, acyl chloride, acyl bromide, acyl iodide, acyl cyanide, acyl azide, ketene, alpha-beta unsaturated ester, alpha-beta unsaturated ketone, alpha-beta unsaturated aldehyde, anhydride, azide, diazo, diazonium, nitrate, nitrate ester, nitroso, nitrile, nitrite, orthoester group, orthocarbonate ester group, O-heterocycle, borane, boronic acid, boronic ester, quinone, dioxirane, diketone and the like. In an example, such surface moieties are disposed on the surface at various reactive active sites of the catalyst.

Carbon Content

In some embodiments, a catalytically-active graphene oxide or graphite oxide catalyst (or other carbon and oxygen-containing catalyst) has a carbon content (mole %) of at least about 25%, or 30%, or 35%, or 40%, or 45%, or 50%, or 55%, or 60%, or 65%, or 70%, or 75%, or 80%, or 85%, or 90%, or 95%, or 99%, or 99.99%. The balance of the catalyst is oxygen, or one or more other surface moieties described herein, or one or more elements selected from the group consisting of hydrogen, oxygen, boron, nitrogen, sulfur, phosphorous, fluorine, chlorine, bromine and iodine. In some embodiments, a graphene oxide or graphite oxide has an oxygen content of at least about 0.01%, or 1%, or 5%, or 15%, or 20%, or 25%, or 30%, or 35%, or 40%, or 45%, or 50%. In some embodiments, a surface-modified graphene oxide or graphite oxide catalyst (MG) has a carbon content of at least about 15%, 20% 25%, 30%, 35%, 40%, 45% or 50% and an oxygen content of at least about 0.001%, 0.01%, 0.1% or 1%. For example, a graphene oxide or graphite oxide catalyst described herein has a carbon content of at least about 25% and an oxygen content of at least about 0.01%. In another example, a graphene oxide or graphite oxide catalyst described herein has a carbon content of at least about 25% and an oxygen content of at least about 1%. In another example, a graphene oxide or graphite oxide catalyst described herein has a carbon content of at least about 50% and an oxygen content of at least about 10%. The oxygen content is measured with the aid of various surface or bulk analytical spectroscopic techniques. As one example, the oxygen content is measured by x-ray photoelectron spectroscopy (XPS) or mass spectrometry (e.g., inductively coupled plasma mass spectrometry, or “ICP-MS”).

In some embodiments, a carbocatalyst has a bulk carbon-to-oxygen ratio of at least about 0.1:1, or 0.5:1, or 1:1, or 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1, or 1,000,000:1. In some cases, a carbocatalyst has a surface carbon-to-oxygen ratio of at least about 0.1:1, or 0.5:1, or 1:1, or 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1, or 1,000,000:1.

pH

In some cases, a heterogeneous catalytically active carbocatalyst (e.g., graphene oxide or graphite oxide catalyst, or other carbon and oxygen-containing catalyst) provides a solution pH of between about 0.1 to about 14 when dispersed in solution. In some cases, a heterogeneous catalytically active carbocatalyst (e.g., graphene oxide or graphite oxide catalyst, or other carbon and oxygen-containing catalyst) provides a reaction solution pH which is acidic (e.g., pH of between about 0.1 to about 6.9) when dispersed in solution. In some cases, a heterogeneous catalytically active carbocatalyst (e.g., graphene oxide or graphite oxide catalyst, or other carbon and oxygen-containing catalyst) provides a reaction solution pH which is basic (e.g., pH of between about 7.1 to about 14) when dispersed in solution. In some cases, a heterogeneous catalytically active carbocatalyst (e.g., graphene oxide or graphite oxide catalyst, or other carbon and oxygen-containing catalyst) provides a reaction solution pH which is neutral (e.g., pH of about 7) when dispersed in solution. By way of example, in one embodiment, “acidic graphene oxide or graphite oxide” that provides a solution pH of 1-3 versus a solution pH of 4-6 is prepared by eliminating the certain optional steps in the material's preparation that involve washing with water. Normally, after the synthesis of a graphene oxide or graphite oxide catalyst is performed in acid, the graphene oxide or graphite oxide is washed with a large volume of water to remove this acid. When the number of wash steps is reduced, a graphene oxide or graphite oxide catalyst with a large amount of exogenous acid adsorbed to its surface is formed and the pH of the solution is lower compared to the pH when the catalyst is prepared by washing the material with water.

In another embodiment, graphene oxide or graphite oxide is basified by exposure to a base. Such a basic graphene oxide or graphite oxide catalyst is prepared by stirring a dispersion of graphene oxide or graphite oxide in water with non-nucleophilic bases such as potassium carbonate or sodium bicarbonate, or organic bases such as pyridine or triethylamine, and isolating the resulting product by filtration. Such carbocatalysts display significantly higher pH values when dispersed in water (e.g., pH=of about 8).

Accordingly, depending on choice of substrates (e.g., whether a starting material is sensitive to acid or base) a suitable carbocatalyst is prepared that provides either an acidic or basic pH upon dispersion in solution.

Stoichiometry and Catalyst Loading

In some embodiments, for any catalytically active carbocatalyst (e.g., graphene oxide or graphite oxide) mediated reaction described herein, e.g., cracking, dehydrogenation, metathesis and/or coupling, the amount of graphene oxide or graphite oxide used is anywhere between 0.01 wt % and 1000 wt %. As used herein, wt % designates weight of the catalyst as compared to the weight of the reactant or reactants. In particular embodiments, the graphene oxide or graphite oxide catalyst may constitute at least 0.01 wt %, between 0.01 wt % and 5 wt %, between 5 wt % and 50 wt %, between 50 wt % and 200 wt %, between 200 wt % and 400 wt %, between 400 wt % and 1000 wt %, or up to 1000 wt %. The amount of catalyst used may vary depending on the type of reaction. For example, reactions in which the catalyst acts on a C—H bond may work well at higher amounts of catalyst, such as up to 400 wt %. Other reactions may work well at lower catalyst levels, such as as little as 0.01 wt %.

In some situations, the groups present at the surface of a catalytically activated carbocatalyst (e.g., a peroxide moiety covalently bound to graphene oxide or graphite oxide) are modified to provide stoichiometric control of a reaction.

Reaction Time

In some embodiments, for any catalytically active carbocatalyst (e.g., graphene oxide or graphite oxide) mediated reaction described herein, e.g., dehydrogenation, cracking, metathesis and/or coupling, the duration of the reaction (e.g., for more than about 10%, about 20%, about 30%, about 40%, about 50%, about 60%, about 70%, about 80%, about 90%, about 95% or about 100% conversion of starting material to product) is from seconds to minutes, from minutes to hours, or from hours to days, or from days to weeks. In one embodiment, for any catalytically active carbocatalyst mediated reaction described herein, the duration of the reaction is from about 1 second to about 5 minutes. In one embodiment, for any catalytically active carbocatalyst mediated reaction described herein, the duration of the reaction is from about 5 minutes to about 30 minutes. In one embodiment, for any catalytically active carbocatalyst mediated reaction described herein, the duration of the reaction is from about 30 minutes to about 60 minutes. In one embodiment, for any catalytically active carbocatalyst mediated reaction described herein, the duration of the reaction is from about 60 minutes to about 4 hours. In one embodiment, for any catalytically active carbocatalyst mediated reaction described herein, the duration of the reaction is from about 4 hours to about 8 hours. In one embodiment, for any catalytically active carbocatalyst mediated reaction described herein, the duration of the reaction is from about 8 hours to about 12 hours. In one embodiment, for any catalytically active carbocatalyst mediated reaction described herein, the duration of the reaction is from about 8 hours to about 24 hours. In one embodiment, for any catalytically active carbocatalyst mediated reaction described herein, the duration of the reaction is from about 24 hours to about 2 days.

In one embodiment, for any catalytically active carbocatalyst mediated reaction described herein, the duration of the reaction is from about 1 day to about 3 days. In one embodiment, for any catalytically active carbocatalyst mediated reaction described herein, the duration of the reaction is from about 1 day to about 5 days. In one embodiment, for any catalytically active carbocatalyst mediated reaction described herein, the duration of the reaction is from about 1 day to about 6 days. In one embodiment, for any catalytically active carbocatalyst mediated reaction described herein, the duration of the reaction is from about 1 day to about 1 week, 2 weeks, 3 weeks or more.

Reaction Temperature

In some embodiments, for any catalytically active carbocatalyst mediated reaction described herein (e.g., dehydrogenation, cracking, metathesis and/or coupling), the reaction is carried out at a temperature between about −78° C., −65° C., −50° C., −25° C., −15° C., −10° C., −5° C., 0° C., 5° C., 10° C., 15° C., 20° C., 25° C., 35° C., 50° C., 60° C., 80° C., and about 25° C., 50° C., 100° C., 150° C., 200° C., 250° C., 300° C., 400° C., 500° C., 600° C., 700° C., 800° C., 900° C., or about 1000° C.

In some embodiments, for any catalytically active carbocatalyst mediated reaction described herein (e.g., dehydrogenation, cracking, metathesis and/or coupling), the reaction is carried out at a temperature between about −78° C. and about 1000° C. In some embodiments, for any catalytically active carbocatalyst mediated reaction described herein (e.g., dehydrogenation, cracking, metathesis and/or coupling), the reaction is carried out at a temperature between about −78° C. and about 800° C. In some embodiments, for any catalytically active carbocatalyst mediated reaction described herein (e.g., e.g., dehydrogenation, cracking, metathesis and/or coupling), the reaction is carried out at a temperature between about −50° C. and about 1000° C. In some embodiments, for any catalytically active carbocatalyst mediated reaction described herein (e.g., dehydrogenation, cracking, metathesis and/or coupling), the reaction is carried out at a temperature between about −50° C. and about 800° C.

In some embodiments, for any catalytically active carbocatalyst mediated reaction described herein (e.g., dehydrogenation, cracking, metathesis and/or coupling), the reaction is carried out at a temperature between about −25° C. and about 1000° C. In some embodiments, for any catalytically active carbocatalyst mediated reaction described herein (e.g., dehydrogenation, cracking, metathesis and/or coupling), the reaction is carried out at a temperature between about −25° C. and about 800° C.

In some embodiments, for any catalytically active carbocatalyst mediated reaction described herein (e.g., dehydrogenation, cracking, metathesis and/or coupling), the reaction is carried out at a temperature between about 0° C. and about 1000° C. In some embodiments, for any catalytically active carbocatalyst mediated reaction described herein (e.g., dehydrogenation, cracking, metathesis and/or coupling), the reaction is carried out at a temperature between about 0° C. and about 800° C. In some embodiments, for any catalytically active carbocatalyst mediated reaction described herein (e.g., dehydrogenation, cracking, metathesis and/or coupling), the reaction is carried out at a temperature between about 0° C. and about 600° C. In some embodiments, for any catalytically active carbocatalyst mediated reaction described herein (e.g., dehydrogenation, cracking, metathesis and/or coupling), the reaction is carried out at a temperature between about 0° C. and about 500° C. In some embodiments, for any catalytically active carbocatalyst mediated reaction described herein (e.g., dehydrogenation, cracking, metathesis and/or coupling), the reaction is carried out at a temperature between about 0° C. and about 450° C. In some embodiments, for any catalytically active carbocatalyst mediated reaction described herein (e.g., dehydrogenation, cracking, metathesis and/or coupling), the reaction is carried out at a temperature between about 0° C. and about 400° C. In some embodiments, for any catalytically active carbocatalyst mediated reaction described herein (e.g., dehydrogenation, cracking, metathesis and/or coupling), the reaction is carried out at a temperature between about 0° C. and about 350° C. In some embodiments, for any catalytically active carbocatalyst mediated reaction described herein (e.g., dehydrogenation, cracking, metathesis and/or coupling), the reaction is carried out at a temperature between about 0° C. and about 300° C. In some embodiments, for any catalytically active carbocatalyst mediated reaction described herein (e.g., dehydrogenation, cracking, metathesis and/or coupling), the reaction is carried out at a temperature between about 0° C. and about 200° C. In some embodiments, for any catalytically active carbocatalyst mediated reaction described herein (e.g., dehydrogenation, cracking, metathesis and/or coupling), the reaction is carried out at a temperature between about 0° C. and about 100° C.

In some embodiments, for any catalytically active carbocatalyst mediated reaction described herein (e.g., dehydrogenation, cracking, metathesis and/or coupling), the reaction is carried out at a temperature between about 25° C. and about 1000° C. In some embodiments, for any catalytically active carbocatalyst mediated reaction described herein (e.g., dehydrogenation, cracking, metathesis and/or coupling), the reaction is carried out at a temperature between about 25° C. and about 800° C. In some embodiments, for any catalytically active carbocatalyst mediated reaction described herein (e.g., dehydrogenation, cracking, metathesis and/or coupling), the reaction is carried out at a temperature between about 25° C. and about 600° C. In some embodiments, for any catalytically active carbocatalyst mediated reaction described herein (e.g., dehydrogenation, cracking, metathesis and/or coupling), the reaction is carried out at a temperature between about 25° C. and about 500° C. In some embodiments, for any catalytically active carbocatalyst mediated reaction described herein (e.g., dehydrogenation, cracking, metathesis and/or coupling), the reaction is carried out at a temperature between about 25° C. and about 450° C. In some embodiments, for any catalytically active carbocatalyst mediated reaction described herein (e.g., dehydrogenation, cracking, metathesis and/or coupling), the reaction is carried out at a temperature between about 25° C. and about 400° C. In some embodiments, for any catalytically active carbocatalyst mediated reaction described herein (e.g., dehydrogenation, cracking, metathesis and/or coupling), the reaction is carried out at a temperature between about 25° C. and about 300° C. In some embodiments, for any catalytically active carbocatalyst mediated reaction described herein (e.g., dehydrogenation, cracking, metathesis and/or coupling), the reaction is carried out at a temperature between about 25° C. and about 200° C. In some embodiments, for any catalytically active carbocatalyst mediated reaction described herein (e.g., dehydrogenation, cracking, metathesis and/or coupling), the reaction is carried out at a temperature between about 25° C. and about 100° C.

In some embodiments, for any catalytically active carbocatalyst mediated reaction described herein (e.g., dehydrogenation, cracking, metathesis and/or coupling), the reaction is carried out at a temperature between about 50° C. and about 1000° C. In some embodiments, for any catalytically active carbocatalyst mediated reaction described herein (e.g., dehydrogenation, cracking, metathesis and/or coupling), the reaction is carried out at a temperature between about 50° C. and about 800° C. In some embodiments, for any catalytically active carbocatalyst mediated reaction described herein (e.g., dehydrogenation, cracking, metathesis and/or coupling), the reaction is carried out at a temperature between about 50° C. and about 600° C. In some embodiments, for any catalytically active carbocatalyst mediated reaction described herein (e.g., dehydrogenation, cracking, metathesis and/or coupling), the reaction is carried out at a temperature between about 50° C. and about 500° C. In some embodiments, for any catalytically active carbocatalyst mediated reaction described herein (e.g., dehydrogenation, cracking, metathesis and/or coupling), the reaction is carried out at a temperature between about 50° C. and about 400° C. In some embodiments, for any catalytically active carbocatalyst mediated reaction described herein (e.g., dehydrogenation, cracking, metathesis and/or coupling), the reaction is carried out at a temperature between about 50° C. and about 300° C. In some embodiments, for any catalytically active carbocatalyst mediated reaction described herein (e.g., dehydrogenation, cracking, metathesis and/or coupling), the reaction is carried out at a temperature between about 50° C. and about 200° C. In some embodiments, for any catalytically active carbocatalyst mediated reaction described herein (e.g., dehydrogenation, cracking, metathesis and/or coupling), the reaction is carried out at a temperature between about 50° C. and about 150° C. In some embodiments, for any catalytically active carbocatalyst mediated reaction described herein (e.g., dehydrogenation, cracking, metathesis and/or coupling), the reaction is carried out at a temperature between about 50° C. and about 100° C.

In some embodiments, for any catalytically active carbocatalyst mediated reaction described herein (e.g., dehydrogenation, cracking, metathesis and/or coupling), the reaction is carried out at a temperature between about 75° C. and about 1000° C. In some embodiments, for any catalytically active carbocatalyst mediated reaction described herein (e.g., dehydrogenation, cracking, metathesis and/or coupling), the reaction is carried out at a temperature between about 75° C. and about 800° C. In some embodiments, for any catalytically active carbocatalyst mediated reaction described herein (e.g., dehydrogenation, cracking, metathesis and/or coupling), the reaction is carried out at a temperature between about 75° C. and about 600° C. In some embodiments, for any catalytically active carbocatalyst mediated reaction described herein (e.g., dehydrogenation, cracking, metathesis and/or coupling), the reaction is carried out at a temperature between about 75° C. and about 500° C. In some embodiments, for any catalytically active carbocatalyst mediated reaction described herein (e.g., dehydrogenation, cracking, metathesis and/or coupling), the reaction is carried out at a temperature between about 75° C. and about 450° C. In some embodiments, for any catalytically active carbocatalyst mediated reaction described herein (e.g., dehydrogenation, cracking, metathesis and/or coupling), the reaction is carried out at a temperature between about 75° C. and about 400° C. In some embodiments, for any catalytically active carbocatalyst mediated reaction described herein (e.g., dehydrogenation, cracking, metathesis and/or coupling), the reaction is carried out at a temperature between about 75° C. and about 300° C. In some embodiments, for any catalytically active carbocatalyst mediated reaction described herein (e.g., dehydrogenation, cracking, metathesis and/or coupling), the reaction is carried out at a temperature between about 75° C. and about 200° C.

In some embodiments, for any catalytically active carbocatalyst mediated reaction described herein (e.g., dehydrogenation, cracking, metathesis and/or coupling), the reaction is carried out at a temperature between about 100° C. and about 1000° C. In some embodiments, for any catalytically active carbocatalyst mediated reaction described herein (e.g., dehydrogenation, cracking, metathesis and/or coupling), the reaction is carried out at a temperature between about 100° C. and about 800° C. In some embodiments, for any catalytically active carbocatalyst mediated reaction described herein (e.g., dehydrogenation, cracking, metathesis and/or coupling), the reaction is carried out at a temperature between about 100° C. and about 600° C. In some embodiments, for any catalytically active carbocatalyst mediated reaction described herein (e.g., dehydrogenation, cracking, metathesis and/or coupling), the reaction is carried out at a temperature between about 200° C. and about 600° C. In some embodiments, for any catalytically active carbocatalyst mediated reaction described herein (e.g., dehydrogenation, cracking, metathesis and/or coupling), the reaction is carried out at a temperature between about 300° C. and about 600° C. In some embodiments, for any catalytically active carbocatalyst mediated reaction described herein (e.g., dehydrogenation, cracking, metathesis and/or coupling), the reaction is carried out at a temperature between about 400° C. and about 600° C. In some embodiments, for any catalytically active carbocatalyst mediated reaction described herein (e.g., dehydrogenation, cracking, metathesis and/or coupling), the reaction is carried out at a temperature between about 200° C. and about 500° C. In some embodiments, for any catalytically active carbocatalyst mediated reaction described herein (e.g., dehydrogenation, cracking, metathesis and/or coupling), the reaction is carried out at a temperature between about 300° C. and about 500° C.

Pressure

In some embodiments, for any catalytically active carbocatalyst mediated reaction described herein (e.g., dehydrogenation, cracking, metathesis and/or coupling), the reaction is carried out at atmospheric pressure. In some embodiments, for any catalytically active carbocatalyst mediated reaction described herein (e.g., dehydrogenation, cracking, metathesis and/or coupling), the reaction is carried out at a pressure of between about 0.1 atm to about 150 atm. In some embodiments, for any catalytically active carbocatalyst mediated reaction described herein (e.g., dehydrogenation, cracking, metathesis and/or coupling), the reaction is carried out at a pressure of between about 1 atm to about 150 atm. In some embodiments, for any catalytically active carbocatalyst mediated reaction described herein (e.g., dehydrogenation, cracking, metathesis and/or coupling), the reaction is carried out at a pressure of between about 5 atm to about 150 atm. In some embodiments, for any catalytically active carbocatalyst mediated reaction described herein (e.g., dehydrogenation, cracking, metathesis and/or coupling), the reaction is carried out at a pressure of between about 10 atm to about 150 atm. In some embodiments, for any catalytically active carbocatalyst mediated reaction described herein (e.g., dehydrogenation, cracking, metathesis and/or coupling), the reaction is carried out at a pressure of between about 20 atm to about 150 atm. In some embodiments, for any catalytically active carbocatalyst mediated reaction described herein (e.g., dehydrogenation, cracking, metathesis and/or coupling), the reaction is carried out at a pressure of between about 50 atm to about 150 atm. In some embodiments, for any catalytically active carbocatalyst mediated reaction described herein (e.g., dehydrogenation, cracking, metathesis and/or coupling), the reaction is carried out at a pressure of between about 100 atm to about 150 atm. In some embodiments, for any catalytically active carbocatalyst mediated reaction described herein (e.g., dehydrogenation, cracking, metathesis and/or coupling), the reaction is carried out at a pressure of between about 1 atm to about 100 atm. In some embodiments, for any catalytically active carbocatalyst mediated reaction described herein (e.g., dehydrogenation, cracking, metathesis and/or coupling), the reaction is carried out at a pressure of between about 5 atm to about 50 atm. In some embodiments, for any catalytically active carbocatalyst mediated reaction described herein (e.g., dehydrogenation, cracking, metathesis and/or coupling), the reaction is carried out at a pressure of between about 10 atm to about 50 atm. In some embodiments, for any catalytically active carbocatalyst mediated reaction described herein (e.g., dehydrogenation, cracking, metathesis and/or coupling), the reaction is carried out at atmospheric pressure. In some embodiments, for any catalytically active carbocatalyst mediated reaction described herein (e.g., dehydrogenation, cracking, metathesis and/or coupling), the reaction is carried out at a pressure of between about 1 atm and about 5 atm. In some embodiments, for any catalytically active carbocatalyst mediated reaction described herein (e.g., dehydrogenation, cracking, metathesis and/or coupling), the reaction is carried out at ambient atmospheric pressure. In some embodiments, for any catalytically active carbocatalyst mediated reaction described herein (e.g., dehydrogenation, cracking, metathesis and/or coupling), the reaction is carried out at a pressure of between about 0.1 atm to about 1 atm. In some embodiments, for any catalytically active carbocatalyst mediated reaction described herein (e.g., dehydrogenation, cracking, metathesis and/or coupling), the reaction is carried out at a pressure of about 1 atm.

Time

In some embodiments, for any catalytically active carbocatalyst mediated reaction described herein (e.g., dehydrogenation, cracking, metathesis and/or coupling), the catalyst is contacted with reactants for a period of time between about 0.01 seconds, or 0.1 seconds, or 1 second, or 10 seconds, or 30 seconds, or 1 minute, or 5 minutes, or 10 minutes, or 15 minutes, or 20 minutes, or 30 minutes, or 1 hour, or 2 hours, or 3 hours, or 4 hours, or 5 hours, or 6 hours, or 12 hours, or 24 hours to about 1 minute, or 5 minutes, or 10 minutes, or minutes, or 20 minutes, or 30 minutes, or 1 hour, or 2 hours, or 3 hours, or 4 hours, or 5 hours, or 6 hours, or 12 hours, or 24 hours, 48 hours, 72 hours, 5 days, 1 week, 2 weeks, 3 weeks, or any suitable length of time.

Oxygenation

In some embodiments, for any catalytically active carbocatalyst mediated reaction described herein (e.g., dehydrogenation, cracking, metathesis and/or coupling), the reaction is carried out under ambient atmosphere. In some embodiments, for any catalytically active carbocatalyst mediated reaction described herein (e.g., dehydrogenation, cracking, metathesis and/or coupling), the reaction is carried out under inert atmosphere. In some embodiments, for any catalytically active carbocatalyst mediated reaction described herein (e.g., dehydrogenation, cracking, metathesis and/or coupling), the reaction is carried out by filing a reaction chamber with a starting material which is a gas.

In further embodiments, for any catalytically active carbocatalyst mediated reaction described herein (e.g., dehydrogenation, cracking, metathesis and/or coupling), the reaction mixture is further oxygenated with an additional oxygen stream, thereby allowing for control of reaction products and/or reaction efficiency and/or conversion ratios. In other embodiments, the reaction mixture is further oxygenated with a sacrificial chemical oxidant such as ozone, hydrogen peroxide, oxone, potassium permanganate, organic peroxides, peroxy acids, perruthenates, lead tetraacetate, chromium oxides, periodates, potassium peroxymonosulfate, sulfur dioxide, chlorates, perchlorates, hypochlorites, perborates, nitrates, nitrous oxide, dinitrogen tetraoxide, silver oxide, osmium tetraoxide, 2,2′-dipyridyldisulfide, ammonium cerium nitrate, benzoquinone, Dess Martin periodinane, a Swern oxidation reagent, molybdenum oxides, pyridine N-oxide, vanadium oxides, TEMPO, potassium ferricyanide, or the like.

The catalytically active carbocatalysts described herein (e.g., graphene and/or graphite oxide) are also prepared with varying extents of oxygenation by changing the amount of oxidant and acid used in the reaction mixture. Variants with decreased oxygen content also possess the desired reactivity. In another aspect, carbocatalysts with variations in oxygenation, including those that are porous and possess high surface areas but have less oxygen functionality, also possess the desired activity. Advantageously, the hydrocarbon hydrocarbon transformations described herein (e.g., conversion of ethane to ethene) are clean compared to other methods and show reduced and/or negligible levels of by-products. Accordingly, contemplated within the scope of embodiments presented herein are modifications to the physical structure of the carbocatalysts, including surface area and porosity, and oxygenation and/or placement of surface moieties to obtain improved reaction efficiencies.

Passes

In some embodiments, for any catalytically active carbocatalyst mediated reaction described herein (e.g., dehydrogenation, cracking, metathesis and/or coupling), the reactant (e.g., a gaseous reactant) is passed over the catalyst once. In alternate embodiments, multiple passes are used to improve conversion efficiency and/or yield of the reaction.

### Catalyst Forms

The carbocatalysts employed in the reactions may be in a variety of physical forms. The catalyst may be powders with particle sizes ranging from 10 nm to 100 μm. The catalyst may be pressed into pellets, rings, honeycomb-like structures, or other forms that may be desirable for packing into a reactor. Physical formation may be accomplished by calcination or sintering of the carbocatalyst on its own. Shaping and molding may also be enhanced by incorporation of polymer binders, organic resins, surfactants, or metallic species. These shaping agents may be included at mass loadings ranging from 0.01 wt % up to 95 wt %. A broad range of polymeric materials and resins may be effective in this role, including polyethylene, polypropylene, poly(ether)s, poly(ether sulfone)s, poly(tetrafluoroethylene), poly(ester)s, poly(urethane)s, poly(amide)s, poly(styrene)s, silicones, Nafion, phenolic resins, and polyelectrolytes or ionomers. The polymers or resins may be acidic, neutral, or basic. Likewise, the surfactants and metallic species may be acidic, neutral, or basic. In further embodiments, the catalytically active carbocatalyst may be coated or deposited on the surface of a reaction vessel.

The available surface area of a catalytically active carbocatalyst available is optionally modified in conjunction with or more passes of reactants to improve conversion efficiency and/or yield of the reaction.

The materials incorporated into the catalyst for the purpose of binding, shaping, and/or molding may exhibit catalytic activity or co-catalytic activity with the carbocatalyst in the desired reaction, or they may be spectator species.

Solvent

In some embodiments, for any catalytically active carbocatalyst mediated reaction described herein (e.g., dehydrogenation, cracking, metathesis and/or coupling), a suitable solvent is any solvent having low reactivity toward the carbocatalyst. In one embodiment, a chlorinated solvent is used, e.g., dichloromethane, chloroform, tetrachloromethane, dichloroethane and the like. In other situations, solvents such as acetonitrile or DMF are used. In some embodiments, water is used as a solvent. Less preferred solvents include solvents such as methanol, ethanol and/or tetrahydrofuran.

In further optional embodiments, the reaction is free of solvent. In another case, a reaction comprises a liquid reactant which is contacted with a catalytically active carbocatalyst as described herein, and the reaction is thereby free of additional solvent. In another case, a reaction comprises a solid reactant which is contacted with a catalytically active carbocatalyst as described herein, wherein upon heating, the solid melts to form a liquid reactant.

Gaseous Phase Reactions

In further embodiments, a reaction comprises a gaseous reactant (e.g., ethane) which is contacted with a heated catalytically active carbocatalyst as described herein. In such instances, a gaseous phase reaction may occur under vacuum, ambient atmospheric pressure, or at elevated pressures (e.g., in a bomb reactor, or a high pressure reactor).

Reactor Systems

In some embodiments, any reaction described herein is a batch reaction. In other embodiments, any reaction described herein is a flow reaction. An exemplary reactor system is described in PCT International Application PCT/US2011/38327 (WO 2011/150325) which disclosure is incorporated herein by reference. It will be understood that any suitable reactor or reaction system may be used in conjunction with the processes and catalytically active carbocatalysts described herein.

In some cases, the reactor is operated under vacuum. In some embodiments, the reactor is operated at a pressure less than about 760 torr, or 1 torr, or 1×10⁻³ ton, or 1×10⁹⁻⁴ torr, or 1×10⁻⁵ torr, or 1×10⁻⁶ ton, or 1×10⁻⁷ ton, or less. In other cases, the reactor 315 is operated at elevated pressures. In some embodiments, the reactor 315 is operated at a pressure of at least about 1 atm, or 2 atm, or 3 atm, or 4 atm, or 5 atm, or 6 atm, or 7 atm, or 8 atm, or 9 atm, or 10 atm, or 20 atm, or 50 atm, or more.

In some embodiments, the reactor is a plug flow reactor, continuous stirred tank reactor, semi-batch reactor or catalytic reactor. In some situations, a catalytic reactor is a shell-and-tube reactor or fluidized bed reactor. In other situations, the reactor includes a plurality of reactors in parallel. This can aid in meeting processing needs while keeping the size of each of the reactors within predetermined limits. For example, if 500 liters/hour of a hydrocarbon is desired but a reactor is capable of providing 250 liters/hour, then two reactors in parallel will meet the desired output of the hydrocarbon. In some cases a flow reactor is employed to improve conversion as a result of superior interaction of the reactant (e.g., ethane, butane) with the catalyst (with higher concentration leading to increased reaction rates).

In some cases a reactor is any one of a flow reactor, batch reactor, a semi-batch reactor, or a discontinuous reactor. In some embodiments, the catalyst bed in a reactor bed is a fixed/packed catalyst bed, of a fluidized catalyst bed. In some embodiments, a reactor is a single pass flow reactor. In other embodiments, a reactor is a recirculated/multiple pass flow reactor. In some instances, flow reactor tube diameters for fixed catalyst beds are about 0.1 inch to 48 inches. In some instances, flow reactor tube diameters for fluidized catalyst beds are about 6 inches to 72 inches. Heating modes for reactors (both flow and batch/semi-batch/discontinuous) may vary. In one embodiment, the reactor tube may be heated from the outside (active heating during gas flow), or a heating column(s) may be placed inside the reactor (active heating during gas flow). In some cases, a catalyst is heated prior to introduction to the reactor (no active heating during gas flow). Flow reactor space velocities are optionally between about 0.001-100 m/s. Separation/purification units are optionally place after each reaction stage.

Turnover

In some embodiments, for any catalytically active carbocatalyst mediated reaction described herein, the turnover number for the reaction is on the order of 10⁻⁵ to about 1,000,000 or greater. In some embodiments, for any catalytically active carbocatalyst mediated reaction described herein, the turnover number for the reaction is on the order of 10⁻⁴ to about 10⁴. In an exemplary embodiment, for any catalytically active carbocatalyst mediated reaction described herein, the turnover number for the reaction is on the order of 10⁻² (expressed in moles of product per mass of catalyst).

Co-Catalyst

In some embodiments, for any catalytically active carbocatalyst mediated reaction described herein, the reaction mixture optionally further comprises a co-catalyst. In one embodiment, such a co-catalyst is, for example, carbon nitride, boron nitride, boron carbon nitride, and the like. In some embodiments, a co-catalyst is an oxidation catalyst (e.g., titanium dioxide, Manganese dioxide). In some embodiments, a co-catalyst is a dehydrogenation catalyst (e.g, Pd/ZnO). In certain embodiments, a co-catalyst is a zeolite.

Co-Reagents

In further optional embodiments, any carbocatalyst mediated reaction described herein is optionally carried out in the presence of co-reagents. In one embodiment, such a co-reagent is an additional oxidizing reagent such as ozone, hydrogen peroxide, oxone, molecular oxygen, or the like, and optionally regenerates the carbocatalyst in situ. In another embodiment, an additional reagent may be a complementary reagent having synergy with the procedures described herein such as a Dess Martin periodinane reagent or a Swern oxidation reagent. In a further embodiment, a co-reagent soaks up a reaction by-product (e.g., hydrogen).

### Co-Catalysts and Catalysts Supported on Graphite Oxide and Catalysts Operated in the Presence of Graphite Oxide or Other Carbocatalysts:

Graphene oxide or graphite oxide and other carbocatalysts are expected to be active when used in conjunction with other catalytic molecules or materials. The catalysts may be metal-containing, organic, inorganic, or macromolecular, and may operate via disparate or identical reaction mechanisms operative in graphene oxide- or graphite oxide-based catalysis. The catalyst may supported on graphene oxide or graphite oxide via chemisorption (e.g., through a ligation interaction with the chemical functionality present on graphene oxide or graphite oxide) or physisorption. The catalysts (either graphene oxide or graphite oxide or the added species) may be enhanced through cooperative chemical effects between graphene oxide or graphite oxide and the catalysts, or may benefit from graphene oxide or graphite oxide's high surface area and available reactive sites. Metal-containing, organic, inorganic, or macromolecular catalysts may also be used in the presence of graphene oxide or graphite oxide, where the two have no interaction and the graphene oxide or graphite oxide operates solely as a spectator species. The catalyst may retain its inherent reactivity and be unaffected by the presence of the graphene oxide or graphite oxide.

### Graphite Intercalation Compounds as Catalysts:

Graphene oxide or graphite oxide and other carbocatalysts are expected to be active in the formation of intercalation compounds (ICs). When formed from graphite-based materials, these materials are known as graphite intercalation compounds (GICs). ICs and GICs are formed through the insertion of a small molecule or polymer into the interlayer region of the stacked structure of graphite and other similar carbon materials. The intercalants may be metallic (e.g., metal salts, coordination complexes), organic (e.g., aryl or aliphatic species), inorganic (e.g., mineral acids), or macromolecules and exhibit diverse chemical properties such as ionic character, various functional groups, and various physical states (i.e., gas, liquid, solid). These ICs and GICs may be reactive, either catalytically or stoichiometrically, and may be considered non-covalently functionalized carbocatalysts. The reactivity of the GIC may be a result of the carbon material itself or the intercalant, or the combination thereof. Though the carbon material or intercalant may enhance the inherent reactivity of the other, either the carbon material of the intercalant may also be an inert spectator species.

### Carbocatalyst Catalyzed Transformations

Catalytically active carbocatalysts comprising surface-modified graphene oxide or graphite oxide are used for activation of unactivated substrates (e.g., hydrocarbons). In these reactions, graphene oxide or graphite oxide exerts its catalytic effect through one or more of exemplary properties such as acidic properties, oxidative properties, dehydrogenation properties, redox properties, or any combination thereof.

### Dehydrogenation

Graphene oxide or graphite oxide is used to catalyze dehydrogenation reactions, such as dehydrogenation of alkanes to alkenes, or dehydrogenation of alkenes to alkynes. This dehydrogenation chemistry is useful when combining this reaction with other reactions.

In specific embodiments, graphene oxide or graphite oxide is used to catalyze dehydrogenation reactions of alkanes to alkenes, including dehydrogenation of straight chain and linear alkanes. In further embodiments, graphene oxide or graphite oxide is used to catalyze dehydrogenation reactions of alkenes to alkynes, including dehydrogenation of terminal or non-terminal alkenes. In other embodiments, graphene oxide or graphite oxide is used to catalyze dehydrogenation reactions of saturated or partially saturated cyclic alkanes (e.g., cyclohexane, methylcyclohexane, and the like) to aromatic compounds (e.g., benzene, toluene, and the like).

In a further aspect, perturbation of the electronic properties of substrates (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to dehydrogenation in the presence of carbo catalysts described herein.

Thus the methods of dehydrogenation in the presence of catalytically active carbo catalysts presented herein have wide synthetic utility.

In exemplary embodiments, dehydrogenation reaction is carried out at a temperature of between 100° C. and 600° C. for a duration of 6-48 hours, in the presence of less than 1 wt % to 1000 wt % graphene oxide or graphite oxide.

It will be recognized that any combination of catalyst loadings, reaction times, solvent and reaction temperatures, as described in the sections above, are contemplated as within the scope of any of the embodiments described above or below.

### Kinetic Control of Hydrocarbon Dehydrogenation Reactions

[chemical structure] [0254] R is H, or any hydrocarbon radical, or two R radicals form a cycle.

In one aspect, recognized herein is the importance of reaction kinetics. In one aspect, the extent of conversion of an alkane (e.g., ethane) to an alkene (e.g., ethene) varies with reaction time and temperature. By way of example, for the conversion of an alkane to an alkene using the processes described herein, after 1 h, approximately 1% alkene is observed, relative to the amount of unreacted alkane in the crude mixture. The conversion increases with time demonstrating that equilibrium has not been established. By way of example, at 168 hours, approximately 10% alkene is observed in the crude NMR spectrum. There appears to be no loss in catalyst reactivity from prolonged heating and no byproducts are observed.

Where the conversion of an alkane to an alkene continues to increase with time, reaction equilibrium has not been established. Accordingly, recognized herein is the importance of temperature. If the reaction temperature is lowered compared to previously used processes, the reaction is under kinetic control, the forward reaction is favored and a higher conversion to the dehydrogenated product is feasible. By contrast, employing other catalysts and/or higher reaction temperature shows a decrease in conversion as reaction time increases, i.e., the reverse reaction is favored at a higher temperature and the thermodynamic product is the alkane, which accounts for the reduced conversion with longer reaction times using previously known methods.

Accordingly, recognized herein is the importance of kinetic control of hydrocarbon dehydrogenations. Accordingly, provided herein are processes that utilize lower temperatures and/or lower pressures compared to other methods (e.g., temperature less than 600° C., or less than 500° C., and pressure of about 1 atmosphere, or between about 1-5 atmosphere), and reaction durations of days, or weeks, to obtain the kinetically favored product of the forward reaction, i.e., the dehydrogenated hydrocarbon.

### Ethane to Ethene

In one embodiment, provided herein is a process for converting ethane to ethene comprising contacting ethane with a catalytically active carbocatalyst (e.g., any carbocatalyst described herein (e.g., graphene and/or graphite oxide) (e.g., graphene and/or graphite oxide)). A suitable reaction temperature and/or pressure is selected for the gaseous reaction.

In one embodiment, the starting material is high purity ethane. In such cases, the reaction product is a high purity ethene. In an alternate embodiment, a low purity ethane (e.g., a mixture of one or more low molecular weight gases such as methane, ethane, propane and/or butane) is used for the reaction. In such cases, the reaction product comprises a mixture of one or more alkenes, including ethene which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of ethane to ethene is effected at a temperature of about 200° C. to about 450° C. and pressure of about 1 atmosphere to about 5 atmospheres for a period of about 10 minutes to about 60 minutes, or longer, such as for 8 hours, 1 day, 4 days, 1 week, or two weeks.

### Butane to 1-Butene

In one embodiment, provided herein is a process for converting butane to 1-butene comprising contacting butane with a catalytically active carbocatalyst (e.g., any carbocatalyst described herein (e.g., graphene and/or graphite oxide) (e.g., graphene and/or graphite oxide)). A suitable reaction temperature and/or pressure is selected for the gaseous reaction.

In one embodiment, the starting material is high purity butane. In such cases, the reaction product is a high purity 1-butene. In an alternate embodiment, a low purity butane (e.g., a mixture of one or more low molecular weight gases such as methane, ethane, propane and/or butane) is used for the reaction. In such cases, the reaction product comprises a mixture of one or more alkenes, including 1-butene which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of butane to 1-butene is effected at a temperature of about 200° C. to about 600° C. and pressure of about 1 atmosphere to about 20 atmospheres for a period of about 1 minute to about 60 minutes, or longer, such as for 8 hours, 1 day, 4 days, 1 week, or two weeks.

### Butane to 2-Butene

In one embodiment, provided herein is a process for converting butane to 2-butene comprising contacting butane with a catalytically active carbocatalyst (e.g., any carbocatalyst described herein (e.g., graphene and/or graphite oxide)). A suitable reaction temperature and/or pressure is selected for the gaseous reaction.

In one embodiment, the starting material is high purity butane. In such cases, the reaction product is a high purity 2-butene. In an alternate embodiment, a low purity butane (e.g., a mixture of one or more low molecular weight gases such as methane, ethane, propane and/or butane) is used for the reaction. In such cases, the reaction product comprises a mixture of one or more alkenes, including 2-butene which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of butane to 2-butene is effected at a temperature of about 200° C. to about 600° C. and pressure of about 1 atmosphere to about 20 atmospheres for a period of about 1 minute to about 60 minutes, or longer, such as for 8 hours, 1 day, 4 days, 1 week, or two weeks.

### Butane to Butadiene

In one embodiment, provided herein is a process for converting butane to butadiene comprising contacting butane with a catalytically active carbocatalyst (e.g., any carbocatalyst described herein (e.g., graphene and/or graphite oxide)). A suitable reaction temperature and/or pressure is selected for the gaseous reaction.

In one embodiment, the starting material is high purity butane. In such cases, the reaction product is a high purity butadiene. In an alternate embodiment, a low purity butane (e.g., a mixture of one or more low molecular weight gases such as methane, ethane, propane and/or butane) is used for the reaction. In such cases, the reaction product comprises a mixture of one or more alkenes, including butadiene which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of butane to butadiene is effected at a temperature of about 200° C. to about 600° C. and pressure of about 1 atmosphere to about 20 atmospheres for a period of about 1 minute to about 60 minutes, or longer, such as for 8 hours, 1 day, 4 days, 1 week, or two weeks.

### Alkane-Alkane Coupling

### Methane to Propane

In one embodiment, provided herein is a process for converting methane to propane comprising contacting methane with a catalytically active carbocatalyst (e.g., any carbocatalyst described herein (e.g., graphene and/or graphite oxide)). A suitable reaction temperature and/or pressure is selected for the gaseous reaction.

In one embodiment, the starting material is high purity methane. In such cases, the reaction product is a high purity propane. In an alternate embodiment, a low purity methane (e.g., a mixture of one or more low molecular weight gases such as methane, ethane, propane and/or butane) is used for the reaction. In such cases, the reaction product comprises a mixture of one or more alkanes, including propane, which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of methane to propane is effected at a temperature of about 400° C. to about 800° C. and pressure of about 1 atmosphere to about 20 atmospheres for a period of about 1 minute to about 60 minutes, or longer, such as for 8 hours, 1 day, 4 days, 1 week, or two weeks.

### Methane to Butane

In one embodiment, provided herein is a process for converting methane to butane comprising contacting methane with a catalytically active carbocatalyst (e.g., any carbocatalyst described herein (e.g., graphene and/or graphite oxide)). A suitable reaction temperature and/or pressure is selected for the gaseous reaction.

In one embodiment, the starting material is high purity methane. In such cases, the reaction product is a high purity butane. In an alternate embodiment, a low purity methane (e.g., a mixture of one or more low molecular weight gases such as methane, ethane, propane and/or butane) is used for the reaction. In such cases, the reaction product comprises a mixture of one or more alkanes, including butane, which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of methane to butane is effected at a temperature of about 400° C. to about 800° C. and pressure of about 1 atmosphere to about 20 atmospheres for a period of about 1 minute to about 60 minutes, or longer, such as for 8 hours, 1 day, 4 days, 1 week, or two weeks.

### Ethane to Butane

In one embodiment, provided herein is a process for converting ethane to butane comprising contacting ethane with a catalytically active carbocatalyst (e.g., any carbocatalyst described herein (e.g., graphene and/or graphite oxide)). A suitable reaction temperature and/or pressure is selected for the gaseous reaction.

In one embodiment, the starting material is high purity ethane. In such cases, the reaction product is a high purity butane. In an alternate embodiment, a low purity ethane (e.g., a mixture of one or more low molecular weight gases such as methane, ethane, propane and/or butane) is used for the reaction. In such cases, the reaction product comprises a mixture of one or more alkanes, including butane, which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of ethane to butane is effected at a temperature of about 400° C. to about 800° C. and pressure of about 1 atmosphere to about 20 atmospheres for a period of about 1 minute to about 60 minutes, or longer, such as for 8 hours, 1 day, 4 days, 1 week, or two weeks.

### Alkane Metathesis

Graphene oxide or graphite oxide and other carbocatalysts are active in the coupling of various alkanes. For example, methane gas reacts with graphene oxide or graphite oxide to form higher order alkanes, such as pentane, heptane, octane, or decane. The products of alkane coupling include, but are not limited to, linear and branched alkanes, e.g., neopentane, isopentane, n-pentane, and the like. In some embodiments, alkanes are catalyzed by graphene oxide or graphite oxide and other carbocatalysts to form polymeric materials, e.g., methane is converted to poly(ethylene). The alkane starting materials for these coupling reactions include gaseous, liquid, and solid alkanes, e.g., methane, ethane, propane, butane, pentane, hexane, heptane, octadecane, nonadecane, and the like. The products of these coupling reactions include, but are not limited to, higher order alkanes, unfunctionalized alkanes, and poly(alkanes), as shown in the top line of the scheme below.

[chemical structure]

In a further embodiment, provided herein is a method for coupling and/or metathesis of an organic compound, comprising:
(a) contacting the organic compound with a catalytically active carbocatalyst described herein (e.g., graphene and/or graphite oxide); and
(b) transforming the organic compound with the aid of the catalytically active carbocatalyst to form a coupling and/or metathesis product.

In some embodiments, the catalytically active carbocatalyst is an oxidized form of graphite. In some embodiments, the catalytically active carbocatalyst is graphene oxide or graphite oxide. In some embodiments, the organic compound comprises an alkane. In some embodiments, the alkane comprises a linear alkane. In some embodiments, the alkane comprises a branched alkane. In some embodiments, the alkane comprises methane, ethane, propane, butane, pentane, hexane, heptane, octadecane, or nonadecane. In some embodiments, the alkane comprises an isomer of butane, pentane, hexane, heptane, octadecane, or nonadecane. In some embodiments, the coupling product comprises an alkane. In some embodiments, the coupling product comprises a linear or branched alkane. In some embodiments, the coupling product comprises a poly(alkane).

### Ethane to Propane

In one embodiment, provided herein is a process for metathesis comprising converting ethane to propane comprising contacting ethane with a catalytically active carbocatalyst (e.g., any carbocatalyst described herein (e.g., graphene and/or graphite oxide)). A suitable reaction temperature and/or pressure is selected for the gaseous reaction.

In one embodiment, the starting material is high purity ethane. In such cases, the reaction product is a high purity propane. In an alternate embodiment, a low purity ethane (e.g., a mixture of one or more low molecular weight gases such as methane, ethane, propane and/or butane) is used for the reaction. In such cases, the reaction product comprises a mixture of one or more alkanes (e.g., propane and methane), including propane, which is further purified using standard procedures.

The reaction is carried out by contacting the gaseous reactants with a graphene oxide or graphite oxide carbocatalyst as described herein. The conversion efficiency is controlled by varying the temperature, pressure and/or catalyst loading, as described in more detail herein. By way of example, in some cases, the conversion of methane to propane is effected at a temperature of about 400° C. to about 800° C. and pressure of about 1 atmosphere to about 20 atmospheres for a period of about 1 minute to about 60 minutes, or longer, such as for 8 hours, 1 day, 4 days, 1 week, or two weeks.

### Cracking

### Hexane to Methane

In one embodiment, provided herein is a process for converting hexane to methane comprising contacting hexane with a catalytically active carbocatalyst (e.g., any carbocatalyst described herein (e.g., graphene and/or graphite oxide)). A suitable reaction temperature and/or pressure is selected for the reaction.

In one embodiment, the starting material is high purity hexane. In such cases, the reaction product is a high purity methane gas and/or a mixture of low molecular weight hydrocarbons. In an alternate embodiment, a low purity hexane (e.g., a mixture of one or more low molecular weight alkanes and/or alkenes) is used for the reaction. In such cases, the reaction product comprises a mixture of one or more gases, including methane, which is further purified using standard procedures.

The reaction is carried out by contacting the reactants with a graphene oxide or graphite oxide carbocatalyst as described herein. The conversion efficiency is controlled by varying the temperature, and/or catalyst loading, as described in more detail herein. By way of example, in some cases, the conversion of hexane to methane is effected at a temperature of about 400° C. to about 800° C. or about 1000° C. and pressure of about 1 atmosphere to about 20 atmospheres for a period of about 1 minute to about 60 minutes, or longer, such as for 8 hours, 1 day, 4 days, 1 week, or two weeks.

### Hexane to Ethane

In one embodiment, provided herein is a process for converting hexane to ethane comprising contacting hexane with a catalytically active carbocatalyst (e.g., any carbocatalyst described herein (e.g., graphene and/or graphite oxide)). A suitable reaction temperature and/or pressure is selected for the reaction.

In one embodiment, the starting material is high purity hexane. In such cases, the reaction product is a high purity ethane gas and/or a mixture of low molecular weight hydrocarbons. In an alternate embodiment, a low purity hexane (e.g., a mixture of one or more low molecular weight alkanes and/or alkenes) is used for the reaction. In such cases, the reaction product comprises a mixture of one or more gases, including ethane, which is further purified using standard procedures.

The reaction is carried out by contacting the reactants with a graphene oxide or graphite oxide carbocatalyst as described herein. The conversion efficiency is controlled by varying the temperature, and/or catalyst loading, as described in more detail herein. By way of example, in some cases, the conversion of hexane to ethane is effected at a temperature of about 400° C. to about 800° C. or about 1000° C. and pressure of about 1 atmosphere to about 20 atmospheres for a period of about 1 minute to about 60 minutes, or longer, such as for 8 hours, 1 day, 4 days, 1 week, or two weeks.

### Hexane to Propane

In one embodiment, provided herein is a process for converting hexane to propane comprising contacting hexane with a catalytically active carbocatalyst (e.g., any carbocatalyst described herein (e.g., graphene and/or graphite oxide)). A suitable reaction temperature and/or pressure is selected for the reaction.

In one embodiment, the starting material is high purity hexane. In such cases, the reaction product is a high purity propane gas and/or a mixture of low molecular weight hydrocarbons. In an alternate embodiment, a low purity hexane (e.g., a mixture of one or more low molecular weight alkanes and/or alkenes) is used for the reaction. In such cases, the reaction product comprises a mixture of one or more gases, including propane, which is further purified using standard procedures.

The reaction is carried out by contacting the reactants with a graphene oxide or graphite oxide carbocatalyst as described herein. The conversion efficiency is controlled by varying the temperature, and/or catalyst loading, as described in more detail herein. By way of example, in some cases, the conversion of hexane to propane is effected at a temperature of about 400° C. to about 800° C. or about 1000° C. and pressure of about 1 atmosphere to about 20 atmospheres for a period of about 1 minute to about 60 minutes, or longer, such as for 8 hours, 1 day, 4 days, 1 week, or two weeks.

### Hexane to Butane

In one embodiment, provided herein is a process for converting hexane to butane comprising contacting hexane with a catalytically active carbocatalyst (e.g., any carbocatalyst described herein (e.g., graphene and/or graphite oxide)). A suitable reaction temperature and/or pressure is selected for the reaction.

In one embodiment, the starting material is high purity hexane. In such cases, the reaction product is a high purity butane gas and/or a mixture of low molecular weight hydrocarbons. In an alternate embodiment, a low purity hexane (e.g., a mixture of one or more low molecular weight alkanes and/or alkenes) is used for the reaction. In such cases, the reaction product comprises a mixture of one or more gases, including butane, which is further purified using standard procedures.

The reaction is carried out by contacting the reactants with a graphene oxide or graphite oxide carbocatalyst as described herein. The conversion efficiency is controlled by varying the temperature, and/or catalyst loading, as described in more detail herein. By way of example, in some cases, the conversion of hexane to butane is effected at a temperature of about 400° C. to about 800° C. or about 1000° C. and pressure of about 1 atmosphere to about 20 atmospheres for a period of about 1 minute to about 60 minutes, or longer, such as for 8 hours, 1 day, 4 days, 1 week, or two weeks.

### Cracking and Dehydrogenation

### Hexane to Propene

In one embodiment, provided herein is a process for converting hexane to propene comprising contacting hexane with a catalytically active carbocatalyst (e.g., any carbocatalyst described herein (e.g., graphene and/or graphite oxide)). A suitable reaction temperature and/or pressure is selected for the reaction.

In one embodiment, the starting material is high purity hexane. In such cases, the reaction product is a high purity propene gas and/or a mixture of low molecular weight hydrocarbons. In an alternate embodiment, a low purity hexane (e.g., a mixture of one or more low molecular weight alkanes and/or alkenes) is used for the reaction. In such cases, the reaction product comprises a mixture of one or more gases, including propene, which is further purified using standard procedures.

The reaction is carried out by contacting the reactants with a graphene oxide or graphite oxide carbocatalyst as described herein. The conversion efficiency is controlled by varying the temperature, and/or catalyst loading, as described in more detail herein. By way of example, in some cases, the conversion of hexane to propene is effected at a temperature of about 400° C. to about 800° C. or about 1000° C. and pressure of about 1 atmosphere to about 20 atmospheres for a period of about 1 minute to about 60 minutes, or longer, such as for 8 hours, 1 day, 4 days, 1 week, or two weeks.

### Hexane to Butene or Butadiene

In one embodiment, provided herein is a process for converting hexane to butane or butadiene comprising contacting hexane with a catalytically active carbocatalyst (e.g., any carbocatalyst described herein (e.g., graphene and/or graphite oxide)). A suitable reaction temperature and/or pressure is selected for the reaction.

In one embodiment, the starting material is high purity hexane. In such cases, the reaction product is a high purity butadiene or butadiene gas and/or a mixture of low molecular weight hydrocarbons. In an alternate embodiment, a low purity hexane (e.g., a mixture of one or more low molecular weight alkanes and/or alkenes) is used for the reaction. In such cases, the reaction product comprises a mixture of one or more gases, including butadiene or butadiene, which is further purified using standard procedures.

The reaction is carried out by contacting the reactants with a graphene oxide or graphite oxide carbocatalyst as described herein. The conversion efficiency is controlled by varying the temperature, and/or catalyst loading, as described in more detail herein. By way of example, in some cases, the conversion of hexane to butadiene is effected at a temperature of about 400° C. to about 800° C. or about 1000° C. and pressure of about 1 atmosphere to about 20 atmospheres for a period of about 1 minute to about 60 minutes, or longer, such as for 8 hours, 1 day, 4 days, 1 week, or two weeks.

Reactions with Graphite Oxide/Solid Acid Catalyst Mixtures

Also contemplated within the scope of the embodiments herein are reactions that are catalyzed with a mixture of graphite oxide and a solid acid (e.g. a zeolite, a clay, a pillared clay, or aluminophosphates). The catalytic activity of graphene oxide or graphite oxide is optionally modified and/or improved with the use of a solid acid catalyst as a co-catalyst. The solid acid catalyst in optionally a zeolite catalyst, including a zeolite catalyst selected from faujasite (FAU), zelolite socony mobil-5 (ZSM-5), mordenite (MOR), or ferrierite (FER). Also contemplated within the scope of embodiments presented herein are synthetic analogs of Faujasite, Sodalite, Analcime, Tobermorite, Cancrinite, Clintobermorite and/or zeolites of type X,Y,A,P, from waste alumino-silicate sources, such as, for example zeolites available from Ceramatec (Salt Lake City, Utah). The zeolite catalyst is optionally blended with graphene oxide or graphite oxide in solution or in the solid state. A wide range of zeolite loadings may be used, between about 0.01 to about 1000 wt %. The reaction conditions for reactions catalyzed with a graphene oxide or graphite oxide/solid acid catalyst mixture are similar to the reaction conditions used for the graphene oxide or graphite oxide-catalyzed hydrocarbon transformations described herein (e.g., conversion of ethane to ethene).

### Certain Embodiments

As described above, provided herein are methods for the conversion of:
(a) an alkane starting material to an alkene product (dehydrogenation);
(b) an alkane starting material to one or more higher molecular weight alkane products (coupling);
(c) an alkane starting material to one or more metathesis products (metathesis);
(d) a higher alkane starting material to one or more lower alkane products (cracking);
(e) a cycloalkane starting material to an alkane product or an alkene product or a combination thereof (cracking);
or any combination thereof;
   comprising contacting any starting material in (a)-(e) with a catalytically active carbocatalyst (e.g., catalytically active surface-modified graphene oxide or graphite oxide) to provide the corresponding product in (a)-(e).

(A)

In a specific embodiment, for reaction (a) above (e.g., dehydrogenation of ethane to ethene), the catalytically active carbocatalyst is a catalytically active surface-modified graphene oxide or graphite oxide material characterized by:

(1) one or more FT-IR features at about 3150 cm⁻¹, 1685 cm⁻¹, 1280 cm⁻¹, or 1140 cm⁻¹.

(2) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface.

(3) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface.

(4) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface.

(5) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and at least about 25% carbon and at least about 0.01% oxygen as measured by x-ray photoelectron spectroscopy (XPS).

(6) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and at least about 25% carbon and at least about 0.01% oxygen as measured by x-ray photoelectron spectroscopy (XPS).

(7) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; at least about 25% carbon and at least about 0.01% oxygen as measured by x-ray photoelectron spectroscopy (XPS).

(8) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 1:1.5 as measured by x-ray photoelectron spectroscopy (XPS).

(9) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 1:1.5 as measured by x-ray photoelectron spectroscopy (XPS).

(10) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 1:1.5 as measured by x-ray photoelectron spectroscopy (XPS).

(11) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 5:1 as measured by x-ray photoelectron spectroscopy (XPS).

(12) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 5:1 as measured by x-ray photoelectron spectroscopy (XPS).

(13) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 5:1 as measured by x-ray photoelectron spectroscopy (XPS).

(14) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1.5:1 and about 2:1 as measured by x-ray photoelectron spectroscopy (XPS).

(15) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1.5:1 and about 2:1 as measured by x-ray photoelectron spectroscopy (XPS).

(16) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1.5:1 and about 2:1 as measured by x-ray photoelectron spectroscopy (XPS).

(17) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 2:1 and about 3:1 as measured by x-ray photoelectron spectroscopy (XPS).

(18) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 2:1 and about 3:1 as measured by x-ray photoelectron spectroscopy (XPS).

(19) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 2:1 and about 3:1 as measured by x-ray photoelectron spectroscopy (XPS).

(20) any of (1)-(19) above, and wherein the majority surface modification is a hydrogen peroxide terminated surface.

(21) any of (1)-(19) above, and wherein the majority surface modification is a hydroxyl terminated surface.

(22) any of (1)-(19) above, and wherein the majority surface modification is a carboxyl terminated surface.

(23) any of (1)-(19) above, and wherein the majority surface modification is a epoxide terminated surface.

(24) any of (1)-(19) above, and wherein the majority surface modification is a ether terminated surface.

(25) any of (1)-(19) above, and wherein the majority surface modification is an anhydride terminated surface.

(26) any of (1)-(19) above, and wherein the majority surface modification is a quinone terminated surface.

(27) any of (1)-(19) above, and wherein the majority surface modification is a lactone terminated surface.

(28) any of (1)-(19) above, and wherein the majority surface modification is an aldehyde terminated surface.

(29) any of (1)-(19) above, and wherein the majority surface modification is a ketone terminated surface.

(30) any of (1)-(19) above, and wherein the majority surface modification is an alcohol terminated surface.

(31) any of (1)-(19) above, and wherein the detectable surface modification is a hydrogen peroxide terminated surface.

(32) any of (1)-(19) above, and wherein the detectable surface modification is a hydroxyl terminated surface.

(33) any of (1)-(19) above, and wherein the detectable surface modification is a carboxyl terminated surface.

(34) any of (1)-(19) above, and wherein the detectable surface modification is a epoxide terminated surface.

(35) any of (1)-(19) above, and wherein the detectable surface modification is a ether terminated surface.

(36) any of (1)-(19) above, and wherein the detectable surface modification is an anhydride terminated surface.

(37) any of (1)-(19) above, and wherein the detectable surface modification is a quinone terminated surface.

(38) any of (1)-(19) above, and wherein the detectable surface modification is a lactone terminated surface.

(39) any of (1)-(19) above, and wherein the detectable surface modification is an aldehyde terminated surface.

(40) any of (1)-(19) above, and wherein the detectable surface modification is a ketone terminated surface.

(41) any of (1)-(19) above, and wherein the detectable surface modification is an alcohol terminated surface.

(B)

In a specific embodiment, for reaction (b) above (e.g., coupling of methane and ethane to propane), the catalytically active carbocatalyst is a catalytically active surface-modified graphene oxide or graphite oxide material characterized by:

(1) one or more FT-IR features at about 3150 cm⁻¹, 1685 cm⁻¹, 1280 cm⁻¹, or 1140 cm⁻¹.

(2) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface.

(3) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface.

(4) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface.

(5) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and at least about 25% carbon and at least about 0.01% oxygen as measured by x-ray photoelectron spectroscopy (XPS).

(6) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and at least about 25% carbon and at least about 0.01% oxygen as measured by x-ray photoelectron spectroscopy (XPS).

(7) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; at least about 25% carbon and at least about 0.01% oxygen as measured by x-ray photoelectron spectroscopy (XPS).

(8) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 1:1.5 as measured by x-ray photoelectron spectroscopy (XPS).

(9) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 1:1.5 as measured by x-ray photoelectron spectroscopy (XPS).

(10) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 1:1.5 as measured by x-ray photoelectron spectroscopy (XPS).

(11) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 5:1 as measured by x-ray photoelectron spectroscopy (XPS).

(12) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 5:1 as measured by x-ray photoelectron spectroscopy (XPS).

(13) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 5:1 as measured by x-ray photoelectron spectroscopy (XPS).

(14) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1.5:1 and about 2:1 as measured by x-ray photoelectron spectroscopy (XPS).

(15) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1.5:1 and about 2:1 as measured by x-ray photoelectron spectroscopy (XPS).

(16) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1.5:1 and about 2:1 as measured by x-ray photoelectron spectroscopy (XPS).

(17) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 2:1 and about 3:1 as measured by x-ray photoelectron spectroscopy (XPS).

(18) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 2:1 and about 3:1 as measured by x-ray photoelectron spectroscopy (XPS).

(19) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 2:1 and about 3:1 as measured by x-ray photoelectron spectroscopy (XPS).

(20) any of (1)-(19) above, and wherein the majority surface modification is a hydrogen peroxide terminated surface.

(21) any of (1)-(19) above, and wherein the majority surface modification is a hydroxyl terminated surface.

(22) any of (1)-(19) above, and wherein the majority surface modification is a carboxyl terminated surface.

(23) any of (1)-(19) above, and wherein the majority surface modification is a epoxide terminated surface.

(24) any of (1)-(19) above, and wherein the majority surface modification is a ether terminated surface.

(25) any of (1)-(19) above, and wherein the majority surface modification is an anhydride terminated surface.

(26) any of (1)-(19) above, and wherein the majority surface modification is a quinone terminated surface.

(27) any of (1)-(19) above, and wherein the majority surface modification is a lactone terminated surface.

(28) any of (1)-(19) above, and wherein the majority surface modification is an aldehyde terminated surface.

(29) any of (1)-(19) above, and wherein the majority surface modification is a ketone terminated surface.

(30) any of (1)-(19) above, and wherein the majority surface modification is an alcohol terminated surface.

(31) any of (1)-(19) above, and wherein the detectable surface modification is a hydrogen peroxide terminated surface.

(32) any of (1)-(19) above, and wherein the detectable surface modification is a hydroxyl terminated surface.

(33) any of (1)-(19) above, and wherein the detectable surface modification is a carboxyl terminated surface.

(34) any of (1)-(19) above, and wherein the detectable surface modification is a epoxide terminated surface.

(35) any of (1)-(19) above, and wherein the detectable surface modification is a ether terminated surface.

(36) any of (1)-(19) above, and wherein the detectable surface modification is an anhydride terminated surface.

(37) any of (1)-(19) above, and wherein the detectable surface modification is a quinone terminated surface.

(38) any of (1)-(19) above, and wherein the detectable surface modification is a lactone terminated surface.

(39) any of (1)-(19) above, and wherein the detectable surface modification is an aldehyde terminated surface.

(40) any of (1)-(19) above, and wherein the detectable surface modification is a ketone terminated surface.

(41) any of (1)-(19) above, and wherein the detectable surface modification is an alcohol terminated surface.

(C)

In a specific embodiment, for reaction (c) above (e.g., metathesis of ethane to propane), the catalytically active carbocatalyst is a catalytically active surface-modified graphene oxide or graphite oxide material characterized by:

(1) one or more FT-IR features at about 3150 cm⁻¹, 1685 cm⁻¹, 1280 cm⁻¹, or 1140 cm⁻¹.

(2) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface.

(3) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface.

(4) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface.

(5) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and at least about 25% carbon and at least about 0.01% oxygen as measured by x-ray photoelectron spectroscopy (XPS).

(6) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and at least about 25% carbon and at least about 0.01% oxygen as measured by x-ray photoelectron spectroscopy (XPS).

(7) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; at least about 25% carbon and at least about 0.01% oxygen as measured by x-ray photoelectron spectroscopy (XPS).

(8) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 1:1.5 as measured by x-ray photoelectron spectroscopy (XPS).

(9) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 1:1.5 as measured by x-ray photoelectron spectroscopy (XPS).

(10) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 1:1.5 as measured by x-ray photoelectron spectroscopy (XPS).

(11) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 5:1 as measured by x-ray photoelectron spectroscopy (XPS).

(12) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 5:1 as measured by x-ray photoelectron spectroscopy (XPS).

(13) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 5:1 as measured by x-ray photoelectron spectroscopy (XPS).

(14) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1.5:1 and about 2:1 as measured by x-ray photoelectron spectroscopy (XPS).

(15) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1.5:1 and about 2:1 as measured by x-ray photoelectron spectroscopy (XPS).

(16) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1.5:1 and about 2:1 as measured by x-ray photoelectron spectroscopy (XPS).

(17) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 2:1 and about 3:1 as measured by x-ray photoelectron spectroscopy (XPS).

(18) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 2:1 and about 3:1 as measured by x-ray photoelectron spectroscopy (XPS).

(19) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 2:1 and about 3:1 as measured by x-ray photoelectron spectroscopy (XPS).

(20) any of (1)-(19) above, and wherein the majority surface modification is a hydrogen peroxide terminated surface.

(21) any of (1)-(19) above, and wherein the majority surface modification is a hydroxyl terminated surface.

(22) any of (1)-(19) above, and wherein the majority surface modification is a carboxyl terminated surface.

(23) any of (1)-(19) above, and wherein the majority surface modification is a epoxide terminated surface.

(24) any of (1)-(19) above, and wherein the majority surface modification is a ether terminated surface.

(25) any of (1)-(19) above, and wherein the majority surface modification is an anhydride terminated surface.

(26) any of (1)-(19) above, and wherein the majority surface modification is a quinone terminated surface.

(27) any of (1)-(19) above, and wherein the majority surface modification is a lactone terminated surface.

(28) any of (1)-(19) above, and wherein the majority surface modification is an aldehyde terminated surface.

(29) any of (1)-(19) above, and wherein the majority surface modification is a ketone terminated surface.

(30) any of (1)-(19) above, and wherein the majority surface modification is an alcohol terminated surface.

(31) any of (1)-(19) above, and wherein the detectable surface modification is a hydrogen peroxide terminated surface.

(32) any of (1)-(19) above, and wherein the detectable surface modification is a hydroxyl terminated surface.

(33) any of (1)-(19) above, and wherein the detectable surface modification is a carboxyl terminated surface.

(34) any of (1)-(19) above, and wherein the detectable surface modification is a epoxide terminated surface.

(35) any of (1)-(19) above, and wherein the detectable surface modification is a ether terminated surface.

(36) any of (1)-(19) above, and wherein the detectable surface modification is an anhydride terminated surface.

(37) any of (1)-(19) above, and wherein the detectable surface modification is a quinone terminated surface.

(38) any of (1)-(19) above, and wherein the detectable surface modification is a lactone terminated surface.

(39) any of (1)-(19) above, and wherein the detectable surface modification is an aldehyde terminated surface.

(40) any of (1)-(19) above, and wherein the detectable surface modification is a ketone terminated surface.

(41) any of (1)-(19) above, and wherein the detectable surface modification is an alcohol terminated surface.

(D)

In a specific embodiment, for reaction (d) above (e.g., cracking of hexane to methane and/or ethane and/or propane and/or butane or a combination thereof), the catalytically active carbocatalyst is a catalytically active surface-modified graphene oxide or graphite oxide material characterized by:

(1) one or more FT-IR features at about 3150 cm⁻¹, 1685 cm⁻¹, 1280 cm⁻¹, or 1140 cm⁻¹.

(2) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface.

(3) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface.

(4) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface.

(5) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and at least about 25% carbon and at least about 0.01% oxygen as measured by x-ray photoelectron spectroscopy (XPS).

(6) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and at least about 25% carbon and at least about 0.01% oxygen as measured by x-ray photoelectron spectroscopy (XPS).

(7) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; at least about 25% carbon and at least about 0.01% oxygen as measured by x-ray photoelectron spectroscopy (XPS).

(8) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 1:1.5 as measured by x-ray photoelectron spectroscopy (XPS).

(9) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 1:1.5 as measured by x-ray photoelectron spectroscopy (XPS).

(10) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 1:1.5 as measured by x-ray photoelectron spectroscopy (XPS).

(11) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 5:1 as measured by x-ray photoelectron spectroscopy (XPS).

(12) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 5:1 as measured by x-ray photoelectron spectroscopy (XPS).

(13) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 5:1 as measured by x-ray photoelectron spectroscopy (XPS).

(14) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1.5:1 and about 2:1 as measured by x-ray photoelectron spectroscopy (XPS).

(15) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1.5:1 and about 2:1 as measured by x-ray photoelectron spectroscopy (XPS).

(16) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1.5:1 and about 2:1 as measured by x-ray photoelectron spectroscopy (XPS).

(17) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 2:1 and about 3:1 as measured by x-ray photoelectron spectroscopy (XPS).

(18) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 2:1 and about 3:1 as measured by x-ray photoelectron spectroscopy (XPS).

(19) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 2:1 and about 3:1 as measured by x-ray photoelectron spectroscopy (XPS).

(20) any of (1)-(19) above, and wherein the majority surface modification is a hydrogen peroxide terminated surface.

(21) any of (1)-(19) above, and wherein the majority surface modification is a hydroxyl terminated surface.

(22) any of (1)-(19) above, and wherein the majority surface modification is a carboxyl terminated surface.

(23) any of (1)-(19) above, and wherein the majority surface modification is a epoxide terminated surface.

(24) any of (1)-(19) above, and wherein the majority surface modification is a ether terminated surface.

(25) any of (1)-(19) above, and wherein the majority surface modification is an anhydride terminated surface.

(26) any of (1)-(19) above, and wherein the majority surface modification is a quinone terminated surface.

(27) any of (1)-(19) above, and wherein the majority surface modification is a lactone terminated surface.

(28) any of (1)-(19) above, and wherein the majority surface modification is an aldehyde terminated surface.

(29) any of (1)-(19) above, and wherein the majority surface modification is a ketone terminated surface.

(30) any of (1)-(19) above, and wherein the majority surface modification is an alcohol terminated surface.

(31) any of (1)-(19) above, and wherein the detectable surface modification is a hydrogen peroxide terminated surface.

(32) any of (1)-(19) above, and wherein the detectable surface modification is a hydroxyl terminated surface.

(33) any of (1)-(19) above, and wherein the detectable surface modification is a carboxyl terminated surface.

(34) any of (1)-(19) above, and wherein the detectable surface modification is a epoxide terminated surface.

(35) any of (1)-(19) above, and wherein the detectable surface modification is a ether terminated surface.

(36) any of (1)-(19) above, and wherein the detectable surface modification is an anhydride terminated surface.

(37) any of (1)-(19) above, and wherein the detectable surface modification is a quinone terminated surface.

(38) any of (1)-(19) above, and wherein the detectable surface modification is a lactone terminated surface.

(39) any of (1)-(19) above, and wherein the detectable surface modification is an aldehyde terminated surface.

(40) any of (1)-(19) above, and wherein the detectable surface modification is a ketone terminated surface.

(41) any of (1)-(19) above, and wherein the detectable surface modification is an alcohol terminated surface.

(E)

In a specific embodiment, for reaction (e) above (e.g., cracking of cyclohexane to methane and/or ethane and/or propane or a combination thereof), the catalytically active carbo catalyst is a catalytically active surface-modified graphene oxide or graphite oxide material characterized by:

(1) one or more FT-IR features at about 3150 cm⁻¹, 1685 cm⁻¹, 1280 cm⁻¹, or 1140 cm⁻¹.

(2) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface.

(3) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface.

(4) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface.

(5) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and at least about 25% carbon and at least about 0.01% oxygen as measured by x-ray photoelectron spectroscopy (XPS).

(6) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and at least about 25% carbon and at least about 0.01% oxygen as measured by x-ray photoelectron spectroscopy (XPS).

(7) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; at least about 25% carbon and at least about 0.01% oxygen as measured by x-ray photoelectron spectroscopy (XPS).

(8) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 1:1.5 as measured by x-ray photoelectron spectroscopy (XPS).

(9) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 1:1.5 as measured by x-ray photoelectron spectroscopy (XPS).

(10) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 1:1.5 as measured by x-ray photoelectron spectroscopy (XPS).

(11) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1:1 and about 5:1 as measured by x-ray photoelectron spectroscopy (XPS).

(12) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1:1 and about 5:1 as measured by x-ray photoelectron spectroscopy (XPS).

(13) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1:1 and about 5:1 as measured by x-ray photoelectron spectroscopy (XPS).

(14) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 1.5:1 and about 2:1 as measured by x-ray photoelectron spectroscopy (XPS).

(15) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 1.5:1 and about 2:1 as measured by x-ray photoelectron spectroscopy (XPS).

(16) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 1.5:1 and about 2:1 as measured by x-ray photoelectron spectroscopy (XPS).

(17) one or more FT-IR features at about 3150 cm⁻¹, 1685 cm⁻¹, 1280 cm⁻¹, or 1140 cm⁻¹; and a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, a lactone terminated surface, a quinone terminated surface, an anhydride terminated surface, or an ether terminated surface; and a carbon-to-oxygen ratio for the catalyst between about 2:1 and about 3:1 as measured by x-ray photoelectron spectroscopy (XPS).

(18) one or more FT-IR features at about 3150 cm⁻¹, 1685 cm⁻¹, 1280 cm⁻¹, or 1140 cm⁻¹; and a surface modification comprising one or more of an epoxide-terminated surface, a peroxide terminated surface, a ketone-terminated surface, an aldehyde terminated surface, a carboxyl terminated surface, a hydroxyl terminated surface, an alcohol terminated surface, a quinone terminated surface, or an ether terminated surface; and a carbon-to-oxygen ratio for the catalyst between about 2:1 and about 3:1 as measured by x-ray photoelectron spectroscopy (XPS).

(19) one or more FT-IR features at about 3150 cm⁻¹, 1685 cm⁻¹, 1280 cm⁻¹, or 1140 cm⁻¹; and a surface modification comprising one or more of an epoxide-terminated surface, a ketone-terminated surface, an aldehyde terminated surface, a carboxyl terminated surface, a hydroxyl terminated surface, or an alcohol terminated surface; and a carbon-to-oxygen ratio for the catalyst between about 2:1 and about 3:1 as measured by x-ray photoelectron spectroscopy (XPS).

(20) any of (1)-(19) above, and wherein the majority surface modification is a hydrogen peroxide terminated surface.

(21) any of (1)-(19) above, and wherein the majority surface modification is a hydroxyl terminated surface.

(22) any of (1)-(19) above, and wherein the majority surface modification is a carboxyl terminated surface.

(23) any of (1)-(19) above, and wherein the majority surface modification is a epoxide terminated surface.

(24) any of (1)-(19) above, and wherein the majority surface modification is a ether terminated surface.

(25) any of (1)-(19) above, and wherein the majority surface modification is an anhydride terminated surface.

(26) any of (1)-(19) above, and wherein the majority surface modification is a quinone terminated surface.

(27) any of (1)-(19) above, and wherein the majority surface modification is a lactone terminated surface.

(28) any of (1)-(19) above, and wherein the majority surface modification is an aldehyde terminated surface.

(29) any of (1)-(19) above, and wherein the majority surface modification is a ketone terminated surface.

(30) any of (1)-(19) above, and wherein the majority surface modification is an alcohol terminated surface.

(31) any of (1)-(19) above, and wherein the detectable surface modification is a hydrogen peroxide terminated surface.

(32) any of (1)-(19) above, and wherein the detectable surface modification is a hydroxyl terminated surface.

(33) any of (1)-(19) above, and wherein the detectable surface modification is a carboxyl terminated surface.

(34) any of (1)-(19) above, and wherein the detectable surface modification is a epoxide terminated surface.

(35) any of (1)-(19) above, and wherein the detectable surface modification is a ether terminated surface.

(36) any of (1)-(19) above, and wherein the detectable surface modification is an anhydride terminated surface.

(37) any of (1)-(19) above, and wherein the detectable surface modification is a quinone terminated surface.

(38) any of (1)-(19) above, and wherein the detectable surface modification is a lactone terminated surface.

(39) any of (1)-(19) above, and wherein the detectable surface modification is an aldehyde terminated surface.

(40) any of (1)-(19) above, and wherein the detectable surface modification is a ketone terminated surface.

(41) any of (1)-(19) above, and wherein the detectable surface modification is an alcohol terminated surface.

F

For each embodiment of each of (A), (B), (C), (D), or (E) above, the catalyst is substantially free of any transition metals (e.g., the catalyst comprises less than 0.1% by weight of any transition metal).

For each embodiment of each of (A), (B), (C), (D), or (E), or for each embodiment of each of (A) and (F), (B) and (F), (C) and (F), (D) and (F), or (E) and (F) described above, the catalyst loading is

(1-1) between about 0.1% to about 10% by weight of the weight of the starting material.

(1-2) between about 0.1% to about 1% by weight of the weight of the starting material.

(1-3) between about 0.001% to about 0.1% by weight of the weight of the starting material.

(1-4) between about 10% to about 50% by weight of the weight of the starting material.

(1-5) between about 10% to about 100% by weight of the weight of the starting material.

(1-6) between about 100% to about 500% by weight of the weight of the starting material.

(1-7) between about 100% to about 1000% by weight of the weight of the starting material.

(1-8) between about 1000% to about 10,000% by weight of the weight of the starting material.

For each embodiment of each of (A) and (1-1), (A) and (1-2), (A) and (1-3), (A) and (1-4), (A) and (1-5), (A) and (1-6), (A) and (1-7), (A) and (1-8), (A) and (F) and (1-1), (A) and (F) and (1-2), (A) and (F) and (1-3), (A) and (F) and (1-4), (A) and (F) and (1-5), (A) and (F) and (1-6), (A) and (F) and (1-7), (A) and (F) and (1-8), (B) and (1-1), (B) and (1-2), (B) and (1-3), (B) and (1-4), (B) and (1-5), (B) and (1-6), (B) and (1-7), (A) and (1-8), (B) and (F) and (1-1), (B) and (F) and (1-2), (B) and (F) and (1-3), (B) and (F) and (1-4), (B) and (F) and (1-5), (B) and (F) and (1-6), (B) and (F) and (1-7), (B) and (F) and (1-8), (C) and (1-1), (C) and (1-2), (C) and (1-3), (C) and (1-4), (C) and (1-5), (C) and (1-6), (C) and (1-7), (C) and (1-8), (C) and (F) and (1-1), (C) and (F) and (1-2), (C) and (F) and (1-3), (C) and (F) and (1-4), (C) and (F) and (1-5), (C) and (F) and (1-6), (C) and (F) and (1-7), (C) and (F) and (1-8), (D) and (1-1), (D) and (1-2), (D) and (1-3), (D) and (1-4), (D) and (1-5), (D) and (1-6), (D) and (1-7), (D) and (1-8), (A) and (F) and (1-1), (D) and (F) and (1-2), (D) and (F) and (1-3), (D) and (F) and (1-4), (D) and (F) and (1-5), (D) and (F) and (1-6), (D) and (F) and (1-7), (D) and (F) and (1-8), (E) and (1-1), (E) and (1-2), (E) and (1-3), (E) and (1-4), (E) and (1-5), (E) and (1-6), (E) and (1-7), (E) and (1-8), (E) and (F) and (1-1), (E) and (F) and (1-2), (E) and (F) and (1-3), (E) and (F) and (1-4), (E) and (F) and (1-5), (E) and (F) and (1-6), (E) and (F) and (1-7), or (E) and (F) and (1-8), the reaction is run at temperature of

(2-1) between about −78° C. and room temperature.

(2-2) between about room temperature and about 100° C.

(2-3) between about 100° C.; and about 300° C.

(2-4) between about 200° C.; and about 400° C.

(2-4) between about 300° C.; and about 500° C.

(2-5) between about 400° C.; and about 450° C.

(2-6) between about 400° C.; and about 500° C.

(2-7) between about 400° C.; and about 600° C.

(3-1) In some cases, for each embodiment described above, the reaction is run under a pressure of 1 atm.

(3-2) In some cases, for each embodiment described above, the reaction is run under a pressure of between about 1-5 atm.

(3-3) In some cases, for each embodiment described above, the reaction is run under a pressure of between about 0.1-1 atm.

(3-4) In some cases, for each embodiment described above, the reaction is run under a pressure of between about 1-10 atm.

(3-5) In some cases, for each embodiment described above, the reaction is run under a pressure of between about 1-50 atm.

(4-1) In some cases, for embodiment (3-1) above, the catalytically active carbocatalyst is a catalytically active surface-modified graphene oxide or graphite oxide, and the catalytically active surface-modified graphene oxide or graphite oxide is catalytically active surface-modified graphene oxide.

(4-2) In some cases, for embodiment (3-1) above, the catalytically active carbocatalyst is a catalytically active surface-modified graphene oxide or graphite oxide, and the catalytically active surface-modified graphene oxide or graphite oxide is catalytically active surface-modified graphite oxide.

(4-3) In some cases, for embodiment (3-2) above, the catalytically active carbocatalyst is a catalytically active surface-modified graphene oxide or graphite oxide, and the catalytically active surface-modified graphene oxide or graphite oxide is catalytically active surface-modified graphene oxide.

(4-4) In some cases, for embodiment (3-2) above, the catalytically active carbocatalyst is a catalytically active surface-modified graphene oxide or graphite oxide, and the catalytically active surface-modified graphene oxide or graphite oxide is catalytically active surface-modified graphite oxide.

(4-5) In some cases, for embodiment (3-3) above, the catalytically active carbocatalyst is a catalytically active surface-modified graphene oxide or graphite oxide, and the catalytically active surface-modified graphene oxide or graphite oxide is catalytically active surface-modified graphene oxide.

(4-6) In some cases, for embodiment (3-3) above, the catalytically active carbocatalyst is a catalytically active surface-modified graphene oxide or graphite oxide, and the catalytically active surface-modified graphene oxide or graphite oxide is catalytically active surface-modified graphite oxide.

(4-7) In some cases, for embodiment (3-4) above, the catalytically active carbocatalyst is a catalytically active surface-modified graphene oxide or graphite oxide, and the catalytically active surface-modified graphene oxide or graphite oxide is catalytically active surface-modified graphene oxide.

(4-8) In some cases, for embodiment (3-4) above, the catalytically active carbocatalyst is a catalytically active surface-modified graphene oxide or graphite oxide, and the catalytically active surface-modified graphene oxide or graphite oxide is catalytically active surface-modified graphite oxide.

(4-9) In some cases, for embodiment (3-5) above, the catalytically active carbocatalyst is a catalytically active surface-modified graphene oxide or graphite oxide, and the catalytically active surface-modified graphene oxide or graphite oxide is catalytically active surface-modified graphene oxide.

(4-10) In some cases, for embodiment (3-5) above, the catalytically active carbocatalyst is a catalytically active surface-modified graphene oxide or graphite oxide, and the catalytically active surface-modified graphene oxide or graphite oxide is catalytically active surface-modified graphite oxide.

(5-1) In some cases, for each of (4-1), (4-2), (4-2), (4-3), (4-4), (4-5), (4-6), (4-7), (4-8), (4-9), or (4-10), the reaction is under kinetic control.

(5-2) In some cases, for each of (4-1), (4-2), (4-2), (4-3), (4-4), (4-5), (4-6), (4-7), (4-8), (4-9), or (4-10), the reaction is under thermodynamic control.

Although specific reaction conditions, turnovers and yields are specified herein, the above conditions, turnover and yields may apply to any reaction catalyzed by a catalytically active carbocatalyst including a surface-modified graphene oxide or graphite oxide.

### EXAMPLES

The present invention may be better understood through reference to the following examples. These examples are included to describe exemplary embodiments only and should not be interpreted to encompass the entire breadth of the invention.

¹H and ¹³C NMR data were collected on a Varian Unity INOVA 400 MHz spectrometer. Chemical shifts (δ) are reported in ppm and referenced downfield from (CH₃)₄Si using the residual solvent peak as an internal standard (¹H: CDCl₃, 7.24 ppm, C₆D₆, 7.15 ppm; C₇D₈, 7.09 ppm; ¹³C: CDCl₃, 77.0 ppm; C₆D₆128.0 ppm; C₇D₈, 137.5 ppm). Mass spectra (CI) were obtained with a Karatos MS9 instrument and are reported as m/z (relative intensity). IR spectra were recorded using either a Thermo Scientific Nicolet iS5 system equipped with an iD3 attenuated total reflectance (ATR) attachment (germanium crystal) or a Perkin-Elmer Spectrum BX system in the solid state in KBr.

XPS spectra were recorded using a commercial X-ray photoelectron spectrometer (Kratos Axis Ultra), utilizing a monochromated Al—Kₐₗₚₕₐ X-ray source (1486.5 eV), hydrid optics (employing a magnetic and electrostatic lens simultaneously) and a multi-channel plate and delay line detector coupled to a hemispherical analyzer. The photoelectrons take-off angle was normal to the surface of the sample and 15°, 26°, 38°, 45°, or 52° with respect to the X-ray beam. All spectra were recorded using a single sweep and an aperture slot of 300×700 microns, and high resolution spectra for each element were collected with a pass energy of 20 eV while surveys were collected using 80 eV pass energy. The pressure measured in the analysis chamber was typically 4×10⁻⁹ Torr during data acquisition.

The ToF-SIMS analyses were carried out under static conditions using the IONTOF TOF-SIMS5, 4″ version instrument housed in the Texas Materials Institute at the University of Texas at Austin. This instrument uses a 30 keV Bi₃⁺ primary ion beam extracted from a Bi/Mn alloy source and a 0.5 keV cesium sputtering ion beam produced by a DSC-S gun which allows a maximum energy of 2 keV. Data acquisition was performed on a PC-based workstation running ION-TOF SurfaceLab 6, linked to a PC running the ION-TOF SurfaceLab 6 software and IGOR which were used for data transfer and processing.

### Example 1

### Preparation of Graphene Oxide or Graphite Oxide Catalyst

The graphene oxide or graphite oxide used in some experiments contained in these examples was prepared according to the following method.

A modified Hummers method was used to prepare the graphite oxide. Surface-modified graphene oxide or graphite oxide (MG) is prepared by reacting flake graphite with potassium permanganate (KMnO₄) in concentrated sulfuric acid (H₂SO₄) at 35° C. for a period of 4 h. The reaction mixture is then quenched by dilution in deionized water followed by the addition of aqueous hydrogen peroxide (H₂O₂). The MG is insoluble in this mixture and is recovered by vacuum filtration followed by washing with excess water to remove residual metal salts and acid. Finally, the hygroscopic product is dried under vacuum to remove residual water affording the product as a dark brown powder. The mass balance of the reaction is described in FIG. 1. Optionally, the ratio of graphite to KMMnO₄ and H₂SO₄ may be varied to yield MG with varying extents of oxygenation.

[chemical structure]

### Example 2

### Preparation of Graphite Oxide

A 250 mL reaction flask is charged with natural flake graphite (1.56 g; SP-1 Bay Carbon Inc. or Alfa Aesar [99%; 7-10 μm]), 50 mL of concentrated sulfuric acid, 25 mL fuming nitric acid, and a stir bar, and then cooled in an ice bath. The flask is then charged with NaClO₃ (3.25 g; note: in some cases NaClO₃ is preferable over KClO₃ due to the aqueous insolubility of KClO₄ that may form during the reaction) under stirring. Additional charges of NaClO₃ (3.25 g) are performed every hour for 11 consecutive hours per day. This procedure is repeated for 3 d. The resulting mixture is poured into 2 L deionized water. The heterogeneous dispersion is then filtered through a coarse fitted funnel or a nylon membrane filter (0.2 μm, Whatman) and the isolated material is washed with additional deionized water (3 L) and 6 N HCl (1 L). The filtered solids are collected and dried under high vacuum to provide a product (3.61 g) as a dark brown powder.

### Example 3

### Preparation of Graphene Oxide

A graphene substrate is provided in a reaction chamber. The substrate does not exhibit one or more FT-IR peaks at 3150 cm⁻¹, 1685 cm⁻¹, 1280 cm⁻¹ or 1140 cm⁻¹. Next, plasma excited species of oxygen are directed from a plasma generator into the reaction chamber and brought in contact with an exposed surface of the graphene substrate. The graphene substrate is exposed to the plasma excited species of oxygen until an FT-IR spectrum of the substrate shows one or more peaks at 3150 cm⁻¹, 1685 cm⁻¹, 1280 cm⁻¹ or 1140 cm⁻¹.

### Example 4

### Alkane to Alkene Using Graphene Oxide or Graphite Oxide

[chemical structure]

The as-prepared catalyst powder of Example 1 is used as a catalyst for the conversion of an alkane to an alkene. A 30 mL glass reactor is charged with MG (50 mg; density ˜2 g mL⁻¹) and sealed with a rubber septum. The reactor is then evacuated (10⁻³ Torr) and backfilled with alkane gas to 1 atm of pressure. The reactor is then heated to 400° C. for varying lengths of time (1-168 h). The reaction is quenched by cooling to −78° C. in a dry ice-isopropanol bath, which condenses the mixture of gases. The septum is then removed and 1-2 mL of CDCl₃ is added to dissolve the crude product mixture. The slurry is then syringe filtered to remove the MG, affording a clear and colorless filtrate. Partial evolution of the dissolved gases is observed visually as the solution warms to room temperature, indicating solution saturation. The solution is then analyzed by NMR spectroscopy to determine the product distribution.

Analysis of the reaction mixture by NMR provides information regarding the reaction including presence or absence of impurities and/or by-products.

Optionally the catalyst is recovered at the conclusion of the reaction and reused under identical conditions without loss in conversion efficiency, demonstrating durable catalyst stability and lifetime.

### Example 5

### Ethane to Ethene Using Graphene Oxide or Graphite Oxide

In a typical preparation, a bomb is charged with graphene oxide or graphite oxide, ethane and a magnetic stir bar. The bomb is then sealed under ambient atmosphere and heated at 400° C. for 1 h. The ethene product is isolated using standard procedures.

### Example 6

### Methane to Propane Using Graphene Oxide or Graphite Oxide

In a typical preparation, a bomb is charged with graphene oxide or graphite oxide, methane and a magnetic stir bar. The bomb is then sealed under ambient atmosphere and maintained at room temperature for 7 days. The propane product is isolated using standard procedures.

### Example 7

### Ethane to Propane Using Graphene Oxide or Graphite Oxide

In a typical preparation, a bomb is charged with graphene oxide or graphite oxide, ethane and a magnetic stir bar. The bomb is then sealed under ambient atmosphere and heated at 400° C. for 5 hours. The propane product is isolated using standard procedures.

### Example 8

### Methane to Butane Using Graphene Oxide or Graphite Oxide

In a typical preparation, a bomb is charged with graphene oxide or graphite oxide, ethane and a magnetic stir bar. The bomb is then sealed under ambient atmosphere and heated at 120° C. for 3 hours. The butane product is isolated using standard procedures.

### Example 9

### Ethane to Butane Using Graphene Oxide or Graphite Oxide

In a typical preparation, a bomb is charged with graphene oxide or graphite oxide, ethane and a magnetic stir bar. The bomb is then sealed under ambient atmosphere and heated at 40° C. for 300 hours. The butane product is isolated using standard procedures.

### Example 10

### Hexane to Methane Using Graphene Oxide or Graphite Oxide

In a typical preparation, a bomb is charged with graphene oxide or graphite oxide, hexane and a magnetic stir bar. The bomb is then sealed under ambient atmosphere and heated at 100° C. for 2 hours. The methane product is isolated using standard procedures.

### Example 11

### Hexane to Ethane Using Graphene Oxide or Graphite Oxide

In a typical preparation, a bomb is charged with graphene oxide or graphite oxide, hexane and a magnetic stir bar. The bomb is then sealed under ambient atmosphere and heated at 100° C. for 4 hours. The ethane product is isolated using standard procedures.

### Example 12

### Hexane to Propane Using Graphene Oxide or Graphite Oxide

In a typical preparation, a bomb is charged with graphene oxide or graphite oxide, hexane and a magnetic stir bar. The bomb is then sealed under ambient atmosphere and heated at 100° C. for 6 hours. The propane product is isolated using standard procedures.

### Example 13

### Hexane to Propene Using Graphene Oxide or Graphite Oxide

In a typical preparation, a bomb is charged with graphene oxide or graphite oxide, hexane and a magnetic stir bar. The bomb is then sealed under ambient atmosphere and heated at 400° C. for 80 hours. The propene product is isolated using standard procedures.

### Example 14

### Hexane to Butane Using Graphene Oxide or Graphite Oxide

In a typical preparation, a bomb is charged with graphene oxide or graphite oxide, hexane and a magnetic stir bar. The bomb is then sealed under ambient atmosphere and heated at 50° C. for 10 hours. The butane product is isolated using standard procedures.

### Example 15

### Hexane to Butene or Butadiene Using Graphene Oxide or Graphite Oxide

In a typical preparation, a bomb is charged with graphene oxide or graphite oxide, hexane and a magnetic stir bar. The bomb is then sealed under ambient atmosphere and heated at 500° C. for 20 hours. The butene or butadiene product is isolated using standard procedures.

### Example 16

### Butane to 1-Butene Using Graphene Oxide or Graphite Oxide

In a typical preparation, a bomb is charged with graphene oxide or graphite oxide, hexane and a magnetic stir bar. The bomb is then sealed under ambient atmosphere and heated at 500° C. for 1 min. The 1-butene product is isolated using standard procedures.

### Example 17

### Butane to 2-Butene Using Graphene Oxide or Graphite Oxide

In a typical preparation, a bomb is charged with graphene oxide or graphite oxide, hexane and a magnetic stir bar. The bomb is then sealed under ambient atmosphere and heated at 1000° C. for 30 s. The 2-butene product is isolated using standard procedures.

### Example 18

### Butane to Butadiene Using Graphene Oxide or Graphite Oxide

In a typical preparation, a bomb is charged with graphene oxide or graphite oxide, hexane and a magnetic stir bar. The bomb is then sealed under ambient atmosphere and heated at 800° C. for 5 min. The butadiene product is isolated using standard procedures.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein is employed in practicing the invention. It is intended that the following claims define the scope of the invention and that methods and structures within the scope of these claims and their equivalents be covered thereby.

What is claimed is:

1. A process for converting
(a) an alkane starting material to an alkene product (dehydrogenation);
(b) an alkane starting material to one or more higher molecular weight alkane products (coupling);
(c) an alkane starting material to one or more metathesis products (metathesis);
(d) a higher alkane starting material to one or more lower alkane products (cracking);
(e) a cycloalkane starting material to an alkane product or an alkene product or a combination thereof (cracking);
or any combination thereof;
comprising contacting any starting material in (a)-(e) with a catalytically active surface-modified graphene oxide or graphite oxide to provide the corresponding product in (a)-(e).

2. The process of claim 1, wherein the catalytically active surface-modified graphene oxide or graphite oxide is catalytically active surface-modified graphene oxide.

3. The process of claim 1, wherein the catalytically active surface-modified graphene oxide or graphite oxide is catalytically active surface-modified graphite oxide.

4. The process of claim 1, wherein the catalytically active surface-modified graphene oxide or graphite oxide is characterized by one or more FT-IR features at about 3150 cm⁻¹, 1685 cm⁻¹, 1280 cm⁻¹, or 1140 cm⁻¹.

5. The process of claim 1, wherein the catalytically active surface-modified graphene oxide or graphite oxide has a surface modification comprising one or more of a hydrogen peroxide-terminated surface, an epoxide-terminated surface, a ketone-terminated surface, a diketone terminated surface, an aldehyde terminated surface, a carboxyl terminated surface, a hydroxyl terminated surface, an alcohol terminated surface, an ether terminated surface, a dioxirane terminated surface, a quinone terminated surface, a peroxy acid terminated surface, an ester terminated surface, an anhydride terminated surface or a perester terminated surface.

6. The process of claim 1, wherein the catalytically active surface-modified graphene oxide or graphite oxide has a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, or an ether terminated surface.

7. The process of claim 1, wherein the catalytically active surface-modified graphene oxide or graphite oxide has a surface modification comprising one or more of an epoxide-terminated surface, a ketone-terminated surface, an aldehyde terminated surface, a carboxyl terminated surface, a hydroxyl terminated surface, or an alcohol terminated surface.

8. The process of claim 1, wherein the catalytically active surface-modified graphene oxide or graphite oxide has at least about 25% carbon and at least about 0.01% oxygen as measured by x-ray photoelectron spectroscopy (XPS).

9. The process of claim 1, wherein the carbon-to-oxygen ratio for the catalytically active surface-modified graphene oxide or graphite oxide is between about 1.5:1 and about 1:1.5 as measured by x-ray photoelectron spectroscopy (XPS).

10. The process of claim 1, wherein the carbon-to-oxygen ratio for the catalytically active surface-modified graphene oxide or graphite oxide is between about 1:1 and about 5:1 as measured by x-ray photoelectron spectroscopy (XPS).

11. The process of claim 1, wherein the carbon-to-oxygen ratio for the catalytically active surface-modified graphene oxide or graphite oxide is between about 2:1 and about 3:1 as measured by x-ray photoelectron spectroscopy (XPS).

12. A process for converting an alkane to an alkene under kinetic control comprising contacting an alkane with a catalytically active surface-modified graphene oxide or graphite oxide of any one of claims 1-11 at a reduced temperature compared to a temperature which is used for the conversion of an alkane to an alkene in the presence of a catalyst which is not a catalytically active surface-modified graphene oxide or graphite oxide.

13. The process of claim 12, wherein the reaction provides a higher yield of the alkene and reduces the reverse reaction of hydrogenation of the alkene.

14. A process for converting ethane to ethene comprising contacting ethane with a catalytically active surface-modified graphene oxide or graphite oxide of any one of claims 1-11.

15. A process for converting methane to propane comprising contacting methane with a catalytically active surface-modified graphene oxide or graphite oxide of any one of claims 1-11.

16. A process for converting ethane to propane comprising contacting ethane with a catalytically active surface-modified graphene oxide or graphite oxide of any one of claims 1-11.

17. A process for converting methane to butane comprising contacting methane with a catalytically active surface-modified graphene oxide or graphite oxide of any one of claims 1-11.

18. A process for converting ethane to butane comprising contacting ethane with a catalytically active surface-modified graphene oxide or graphite oxide of any one of claims 1-11.

19. A process for converting hexane to methane comprising contacting hexane with a catalytically active surface-modified graphene oxide or graphite oxide of any one of claims 1-11.

20. A process for converting hexane to ethane comprising contacting hexane with a catalytically active surface-modified graphene oxide or graphite oxide of any one of claims 1-11.

21. A process for converting hexane to propane comprising contacting hexane with a catalytically active surface-modified graphene oxide or graphite oxide of any one of claims 1-11.

22. A process for converting hexane to propene comprising contacting hexane with a catalytically active surface-modified graphene oxide or graphite oxide of any one of claims 1-11.

23. A process for converting hexane to butane comprising contacting hexane with a catalytically active surface-modified graphene oxide or graphite oxide of any one of claims 1-11.

24. A process for converting hexane to butene or butadiene comprising contacting hexane with a catalytically active surface-modified graphene oxide or graphite oxide of any one of claims 1-11.

25. A process for converting butane to 1-butene comprising contacting butane with a catalytically active surface-modified graphene oxide or graphite oxide of any one of claims 1-11.

26. A process for converting butane to 2-butene comprising contacting butane with a catalytically active surface-modified graphene oxide or graphite oxide of any one of claims 1-11.

27. A process for converting butane to butadiene comprising contacting butane with a catalytically active surface-modified graphene oxide or graphite oxide of any one of claims 1-11.

28. A process for alkane metathesis comprising contacting the alkane with a catalytically active surface-modified graphene oxide or graphite oxide of any one of claims 1-11.

29. The process of claim 28, wherein the alkane is a C₁-C₆ alkane and the product obtained from contacting the C₁-C₆ alkane with a catalytically active surface-modified graphene oxide or graphite oxide is a mixture of higher and/or lower C₁-C₁₂ alkanes.

30. A process for dehydrogenation of an alkane comprising contacting the alkane with a catalytically active surface-modified graphene oxide or graphite oxide of any one of claims 1-11.

31. The process of claim 30, wherein the alkane is a C₂-C₆ alkane and the product obtained from contacting the C₂-C₆ alkane with a catalytically active surface-modified graphene oxide or graphite oxide is a mixture of one or more C₂-C₆ alkenes.

32. The process of claim 30, wherein the alkane is a C₄-C₆ alkane and the product obtained from contacting the C₄-C₆ alkane with a catalytically active surface-modified graphene oxide or graphite oxide is a mixture of one or more C₄-C₆ dienes.

33. The process of any one of claims 1-11, wherein the process comprises a solvent-free reaction.

34. The process of any one of claims 1-11, wherein the process comprises one or more gaseous reactants in contact with the catalytically active surface-modified graphene oxide or graphite oxide.

35. The product formed according to the process of any one of claims 1-34.

36. A reaction vessel comprising the starting materials, the product and the catalytically active surface-modified graphene oxide or graphite oxide of any one of claims 1-34.

37. The reaction vessel of claim 36 further comprising a source of heat to heat the reaction vessel to a desired temperature, a device for controlling the temperature of the reaction vessel and a device for determining the temperature within the reaction vessel.

38. The reaction vessel of any one of claims 36 and 37, in the form of a fluidized bed reactor.

39. The reaction vessel of any of one claims 36-38, further comprising a solid acid catalyst.

40. A process for converting
(a) a saturated hydrocarbon moiety on a hydrocarbon starting material to an unsaturated hydrocarbon moiety on a hydrocarbon product (dehydrogenation);
(b) a hydrocarbon starting material to one or more higher molecular weight hydrocarbon products (coupling);
(c) a hydrocarbon starting material to one or more metathesis products (metathesis);
(d) a higher hydrocarbon starting material to one or more lower hydrocarbon products (cracking);
or any combination thereof;
comprising contacting any starting material in (a)-(d) with a catalytically active carbocatalyst to provide the corresponding product in (a)-(d).

41. The process of claim 40, wherein the carbocatalyst is selected from a fullerene-related material, amorphous carbon, crystalline carbon, mesoporous carbon, graphene oxide or graphite oxide derived material, or activated carbon.

42. The process of claim 40, wherein the carbocatalyst is selected from a fullerene-related material, amorphous carbon, crystalline carbon, graphene oxide or graphite oxide derived material, or activated carbon.

43. The process of claim 40, wherein the starting material in (a)-(d) is a material that does not comprise activated C—H bonds.

44. The process of claim 40, wherein the catalytically active carbocatalyst is a modified form of graphene oxide or graphite oxide.

45. The process of claim 40, wherein the catalytically active carbocatalyst is characterized by one or more FT-IR features at about 3150 cm⁻¹, 1685 cm⁻¹, 1280 cm⁻¹, or 1140 cm⁻¹.

46. The process of claim 40, wherein the catalytically active carbocatalyst is a catalytically active surface-modified graphene oxide or graphite oxide.

47. The process of claim 46, wherein the catalytically active surface-modified graphene oxide or graphite oxide is catalytically active surface-modified graphene oxide.

48. The process of claim 46, wherein the catalytically active surface-modified graphene oxide or graphite oxide is catalytically active surface-modified graphite oxide.

49. The process of claim 40, wherein the catalytically active carbocatalyst has a surface modification comprising one or more of a hydrogen peroxide-terminated surface, an epoxide-terminated surface, a ketone-terminated surface, a diketone terminated surface, an aldehyde terminated surface, a carboxyl terminated surface, a hydroxyl terminated surface, an alcohol terminated surface, an ether terminated surface, a dioxirane terminated surface, a quinone terminated surface, a peroxy acid terminated surface, an ester terminated surface, an anhydride terminated surface or a perester terminated surface.

50. The process of claim 40, wherein the catalytically active carbocatalyst has a surface modification comprising one or more of a hydrogen peroxide-terminated surface, an epoxide-terminated surface, a ketone-terminated surface, an aldehyde terminated surface, a carboxyl terminated surface, a hydroxyl terminated surface, an alcohol terminated surface, or an ether terminated surface.

51. The process of claim 40, wherein the catalytically active carbocatalyst has a surface modification comprising one or more of an epoxide-terminated surface, a ketone-terminated surface, an aldehyde terminated surface, a carboxyl terminated surface, a hydroxyl terminated surface, or an alcohol terminated surface.

52. The process of claim 40, wherein the catalytically active carbocatalyst has at least about 25% carbon and at least about 0.01% oxygen as measured by x-ray photoelectron spectroscopy (XPS).

53. The process of claim 40, wherein the carbon-to-oxygen ratio for the catalytically active carbocatalyst is between about 1.5:1 and about 1:1.5 as measured by x-ray photoelectron spectroscopy (XPS).

54. The process of claim 40, wherein the carbon-to-oxygen ratio for the catalytically active carbocatalyst is between about 1:1 and about 5:1 as measured by x-ray photoelectron spectroscopy (XPS).

55. The process of claim 40, wherein the carbon-to-oxygen ratio for the catalytically active carbocatalyst is between about 2:1 and about 3:1 as measured by x-ray photoelectron spectroscopy (XPS).
